(19) United States
(12) Patent Application Publication
(10) Pub. No.: US 2023/0181793 A1
(43) Pub. Date: Jun. 15, 2023

(54) FUNCTIONALIZED POLY(GLYCEROL SEBACATE)S AND USES THEREOF

(71) Applicant: CORNELL UNIVERSITY, Ithaca, NY (US)

(72) Inventors: Xiaochu Ding, Ithaca, NY (US); Yadong Wang, Ithaca, NY (US)

(21) Appl. No.: 17/923,679

(22) PCT Filed: May 17, 2021
(86) PCT No.: PCT/US2021/032740

§ 371 Date: Nov. 7, 2022

Related U.S. Application Data
(60) Provisional application No. 63/025,691, filed on May 15, 2020.

(51) Int. Cl. A61L 27/18; A61L 27/50; C08G 63/16; C08G 63/91

(52) U.S. Cl. CPC A61L 27/18; A61L 27/507; C08G 63/16; C08G 63/916

(57) ABSTRACT

Poly(glycerol sebacate) (PGS) polymers, which may be referred to as a functionalized poly(glycerol sebacate) polymers. The PGS polymers include pendant aliphatic carboxyl ate groups and/or pendant aryl carboxyl ate groups covalently bound to the glycerol group of the glycerol sebacate backbone of the polymer. Polymeric materials including a plurality of glycerol sebacate groups, where at least a portion of the individual glycerol sebacate groups have a pendant aliphatic carboxylate group and/or pendant aryl carboxyl ate group covalently bound to the glycerol group of the glycerol sebacate group. The PGS polymers or polymeric materials may be crosslinked PGS polymers or polymeric materials. The PGS polymers and polymeric materials may be made by post-polymerization functionalization. The PGS polymers and polymeric materials may be in fiber form. A material, which may be a fabric, may include a fiber or plurality of fibers. A material may be used to form a tissue graft.

### CROSS-REFERENCE TO RELATED APPLICATIONS

This application claims the benefit of U.S. Provisional Application No. 63/025,691 filed May 15, 2020. The entire contents of the above-identified application are hereby fully incorporated herein by reference.

### STATEMENT REGARDING FEDERALLY SPONSORED RESEARCH

This invention was made with government support under grant numbers HL089658 awarded by the National Institutes of Health and DMR-1719875 awarded by the National Science Foundation. The government has certain rights in the invention.

### BACKGROUND OF THE DISCLOSURE

Poly(glycerol sebacate) (PGS) elastomer has been intensively studied in biomedical applications such as nerve conduits, heart patches, drug delivery systems and adhesives, among others. Of particular interest, is engineering of a PGS vascular graft for small-diameter arteries. Typically, a synthetic graft is fully remodeled by host cells in months post-implantation. The neo-tissue possesses extracellular matrix (ECM), architecture, and performance resembling to the native arteries, as demonstrated in rat abdominal aorta and carotid models. Compared to the other synthetic grafts made from materials such as PCL, PLLA, PGA, PLGA, PET and ePTFE, the quickly bioresorbable PGS graft did not cause thrombosis, stenosis, infection or calcification. On the other hand, it has been noted that the quick degradation of the PGS graft led to the medial layer of the neo-artery not as mature as the native arteries. This may be likely because the infiltrated cells could not sufficiently proliferate and synthesize ECM prior to the degradation of the PGS scaffold.

To date, many methodologies have been reported to modulate the physicochemical and biological properties of the PGS and introduce other functions for biomedical applications. Examples include acrylation, tyramine-functionalization, urethane crosslinking, copolymerization with other monomers and blends with other materials, among others. For example, among these modified PGS elastomers, the elasticity and modulus could be adjusted by the curing time and crosslinking densities. Less crosslinks typically lead to a softer elastomer but a quicker degradation.

### SUMMARY OF THE DISCLOSURE

In an aspect, the present disclosure provides PGS polymers and polymeric materials. The PGS polymers and polymeric materials comprise pendant aliphatic carboxylate groups and/or pendant aryl carboxylate groups. In various examples, a poly(glycerol sebacate) (PGS) polymer comprises pendant aliphatic carboxylate groups and/or pendant aryl carboxylate groups. Each pendant aliphatic carboxylate group and/or pendant aryl carboxylate group is covalently bound to the glycerol group of the glycerol sebacate backbone of the polymer. The pendant aliphatic carboxylate groups may be saturated aliphatic carboxylate groups, which may be linear saturated aliphatic carboxylate groups, or unsaturated aliphatic carboxylate groups, which may be linear unsaturated aliphatic carboxylate groups. The pendant saturated aliphatic carboxylate groups may be fatty acid carboxylate groups. The pendant saturated aliphatic carboxylate groups, which may be pendant fatty acid carboxylate groups, may be formed from saturated aliphatic carboxylic acids, which may be naturally-occurring fatty acids. A PGS polymer may be pre-polymer that is further crosslinked to form a polymer network. In various examples, a network polymer comprises PGS polymer domains. In various examples, a PGS polymer is at least partially crosslinked.

A polymeric material comprises a plurality of glycerol sebacate groups. At least a portion of the individual glycerol sebacate groups have a pendant aliphatic carboxylate group and/or pendant aryl carboxylate group covalently bound to the glycerol group of the glycerol sebacate group. A polymeric material may be a copolymer. A copolymer may be a block copolymer.

In an aspect, the present disclosure provides compositions. The compositions comprise one or more PGS polymer(s) and/or one or more polymeric material(s). A composition may be a fiber or a plurality of fibers. The fiber(s) comprise one or more PGS polymer(s) and/or one or more polymeric material(s). A fiber may be an electrospun fiber. A fiber or plurality of fibers may be used to form a material, such as, for example, a fabric.

A material comprises one or more PGS polymer(s) and/or polymeric material(s). One or more of the PGS polymer(s) and/or polymeric material(s) may be at least partially crosslinked (or a crosslinked network). A material may comprise a plurality of one or more fiber(s). A material may be a fabric.

A composition may be a tissue graft. In various examples, a tissue graft comprises one or more poly(glycerol sebacate)(s), one or more polymeric material(s), or a combination thereof, and/or a fiber or material comprising one or more poly(glycerol sebacate)(s), one or more polymeric material(s), or a combination thereof. The shape of the functionalized PGS material may also be manipulated for specific tissue engineering applications. A tissue graft may be a vascular graft. A vascular graft may be arterial graft, which may be a small artery graft. A small artery graft may have a lumen diameter of 6 mm or less.

### BRIEF DESCRIPTION OF THE FIGURES

For a fuller understanding of the nature and objects of the disclosure, reference should be made to the following detailed description taken in conjunction with the accompanying figures.

FIG. 1A, 1B1-1B4, and 1C1-1C2 show: (1A) the synthesis for palmitate-functionalized PGS (PPGS); (1B1-1B4) proton NMR analyses identification of PPGS structure and determination of actual palmitate contents, using integral area ratio of Hₐ to Hₑ (FIGS. 2A-2D) relative to PGS repeat units, of (1B1) 2 mol% (PPGS-2), (1B2) 5 mol% (PPGS-5), (1B3) 9 mol% (PPGS-9), and (1B4) 16 mol% (PPGS-16), for PPGS prepared using theoretical palmitate contents of 5, 10, 20 and 40 mol.%, respectively; (1C1-1C2) a schematic illustration of the thermally crosslinked polymer networks: (1C1) PGS networks with representative hydrogen bonds formed between the free hydroxyls and the bound water molecules, and (1C2) PPGS networks with palmitate pendants which affect hydrophobicity, hydrogen bonds and the polymer chain packing.

FIGS. 2A-2D show the proton NMR analyses used to determine that the actual palmitate contents of PPGS pre-polymers prepared using theoretical palmitate contents of 5, 10, 20, and 40 mol.% relative to PGS repeat units: (2A) 2 mol% (PPGS-2), (2B) 5 mol% (PPGS-5), (2C) 9 mol% (PPGS-9) and (2D) 16 mol% (PPGS-16), respectively, as determined according to the integral area ratio of protons Hₐ to Hₑ.

FIG. 3 shows gel permeation chromatography analyses of the PGS and PPGS pre-polymers. The GPC spectra demonstrate that with more palmitic anhydrides in the reactions, the fraction of high molecular weight pre-polymers is increased. PPGS-2 is nearly identical with the PGS control, indicating no significant changes in the molecular weight. Mₙ, M_{w} and PDI of the PPGS pre-polymers increase accordingly with the palmitate contents (Table 1).

FIGS. 4A-4B show: (4A) a modified synthetic scheme for a PPGS pre-polymer using the reaction conditions of FIG. 1A and at a theoretical 40 mol.% of palmitic anhydrides relative to the PGS repeat units, except that the reaction time is increased from 20 h (h = hour(s)) to 32 h; and (4B) the proton NMR analysis used to identify the actual palmitate content in resultant PPGS pre-polymer to be approximately 16 mol.%, similar to the PPGS-16 prepared by 20 h reaction.

FIGS. 5A-5E show thermal and crystalline properties of the elastomers by DSC analysis: (5A) PGS control, (5B) PPGS-2, (5C) PPGS-5 (5D) PPGS-9 and (5E) PPGS-16. The thermal profiles from the cooling and second heating are demonstrated. The enthalpies associated with the thermal events are listed in Table 1. All elastomers demonstrate main thermal events with Tg, Tm and Tc below 10° C., indicating they are semi-crystalline materials and easy to reach a rubbery elastic state at a temperature above 10° C.

FIGS. 6A-6E show representative microscopic morphologies at the cross sections of the elastomers by SEM observation: (6A) PGS control, (B) PPGS-2, (6C) PPGS-5, (6D) PPGS-9 and (6E) PPGS-16, (scale bar, 20 pm). Red arrows mark examples of micro-island morphologies. White arrow indicates another type of self-assembled micro-patterns existing in the PPGS-9 elastomers. The bright particles or debris are formed from the cutting process when preparing the cross sections by a blade.

FIGS. 7A-7B show representative microscopic morphologies at the cross sections by SEM observation: (7A) PPGS-Mix-9 and (7B) PPGS-Mix-16 (scale bar, 20 µm). The PPGS-Mix-9 and PPGS-Mix-16 elastomers were made by physically mixing PGS pre-polymer with 9 mol.% and 16 mol.% of free palmitic acid respectively, followed by the same crosslinking conditions used to make the PPGS elastomers. Few micro-islands or micro-patterns are observed in the two elastomers. The waving structures dominate the morphologies at their cutting interfaces. The results indicate that physical mixture of the palmitic acid with the PGS could not effectively alter the microstructures inside the two elastomers.

FIGS. 8A1-8A5 and 8B1-8B4 show: (8A1-8A5) representative cyclic tensile tests to evaluate the elasticity of elastomers: (8A1) PGS control, (8A2) PPGS-2, (8A3) PPGS-5, (8A4) PPGS-9 and (8A5) PPGS-16 (n = 3). All elastomers sustained reversible elastic deformations for at least 1000 cycles without failure. Little hysteresis loop was observed in the PPGS-9 and PPGS-16 samples, indicating improved elastic recovery for the palmitate pendants at 9 and 16 mol.%; and (8B1-8B4) comparison of (8B 1) representative stress-strain curves, (8B2) strain at break (ns, p = 0.1222), (8B3) UTS (***, p < 0.0001) and (8B4) E (***, p < 0.0001). As the palmitate contents increase, the PPGS elastomers show reduced UTS and E values; the strain at break demonstrates a slight increase, but not statistically different, compared to the PGS control. One-way ANOVA analysis with Bonferroni’s multiple comparison test is performed for statistical analysis. p < 0.05 is considered significantly different. The data represent mean value ± SD (n = 5).

FIGS. 9A-9D show comparison of (9A) representative stress-strain curves, (9B) strain at break, (9C) UTS and (9D) E of the PGS control, PPGS-Mix-9 and PPGS-Mix-16 elastomers as measured by uniaxial tensile tests (n = 5). The data indicate that physically mixing palmitic acid with the PGS pre-polymer could not significantly alter the mechanical properties of the elastomers.

FIGS. 10A-10D show: (10A) the hydrophobicity of the PPGS and PGS elastomers as determined by water contact angle measurements (n = 5) (***, p < 0.0001); (10B) the accelerated degradation of the elastomers in a 60 mM NaOH solution at 37° C. over 120 h (n = 4). For each time point, the degradations of the five elastomers were statistically analyzed. The results are: 6 h, **, p = 0.0025; 12 h, ***, p < 0.0001; 18 h, ***, p < 0.0001; 24 h, ***, p = 0.0006; 48 h, 72 h and 120 h, ***, p < 0.0001; the degradation of porous PPGS and PGS elastomeric scaffolds (10C) in the 60 mM NaOH solution at 37° C. over 18 h (n = 6, * * *, p = 0.0002) and (10D) in PBS (pH 7.4) over one month (n = 3, ns, p = 0.9138). One-way ANOVA analysis with Bonferroni’s multiple comparison test is performed for all statistical analyses. p < 0.05 is considered significantly different. The data represent mean value ± SD.

FIGS. 11A1-11A5, 11B, 11C1-11C2, and 11D1-11D2 show: (11A1-11A5) degradation tests of the PPGS and PGS control elastomers were performed in 60 mM NaOH solution at 37° C. for 72 h. (11A1) PGS, (11A2) PPGS-2, (11A3) PPGS-5, (11A4) PPGS-9 and (11A5) PPGS-16; (11B) Palmitate contents released from these elastomers were calculated for comparison; (11C1-2): morphologies of the PGS sample (11C1) before and (11C2) after degradation for 72 h; (11D1-11D2): morphologies of the PPGS-16 sample (11D1) before and (11D2) after degradation for 72 h.

FIGS. 12A-12G show: (12A) MTT and live/dead assays to evaluate the metabolic activity and viability of HLTVECs on the PPGS and PGS elastomers over 48 h (n = 4). TCPS is used as a control. One-way ANOVA analysis with Bonferroni’s multiple comparison test is performed for statistical analyses. MTT assay, ns, p = 0.8781; live/dead assay, ns, p = 0.9814. p < 0.05 is considered significantly different. (12B-12G) Live/dead fluorescent images of HLTVECs cultured on the elastomers; (12B) TCPS control, (12C) PGS, (12D) PPGS-2, (12E) PPGS-5, (12F) PPGS-9 and (12G) PPGS-16. Scale bar: 100 µm. Compared to the TCPS and PGS controls, the MTT and viability assays show no significant differences among them. The cell attachment, spread and morphologies on these elastomers remained similar to the TCPS control, except the cell densities decreased slightly along with the palmitate contents.

### DETAILED DESCRIPTION OF THE DISCLOSURE

Although subject matter herein is described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range.

As used herein, unless otherwise stated, the term “bioactive agent(s)” refers to compound(s) or entit(ies) that alter, inhibit, activate, or otherwise affect biological or chemical events. For example, bioactive agents include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, antipyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents, and the like, and combinations thereof. In certain examples, a bioactive agent is a drug.

As used herein, unless otherwise stated, the term “biocompatible” refers is a PGS polymer, polymeric material, or composition that can be substantially non-toxic in the in vivo environment of its intended use, and is not substantially rejected by the patient’s physiological system (e.g., is nonantigenic). This may be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material’s toxicity, infectivity, pyrogenicity, irritation potential, reactivity, hemolytic activity, carcinogenicity and/or immunogenicity.

As used herein, unless otherwise stated, the term “biomolecules” refers to molecules (e.g., proteins, amino acids, peptides, polynucleotides, nucleotides, carbohydrates, sugars, lipids, nucleoproteins, glycoproteins, lipoproteins, steroids, and the like) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as, for example growth factors and chemotactic factors, antibodies, vaccines, haptens, toxins, interferons, ribozymes, anti-sense agents, plasmids, DNA, RNA, and the like.

As used herein, unless otherwise stated, the term “biodegradable polymer” refers to a PGS polymer or polymeric material that can be cleaved either enzymatically or hydrolytically to break it down sufficiently so as to allow the body to absorb or clear it away. In certain examples, a biodegradable PGS polymer/polymeric material is a PGS polymer/polymeric material that degrades fully (i.e., down to monomeric species) under physiological or endosomal conditions. In various examples, a biodegradable vascular graft is a graft in which at least a significant portion (such as, for example at least 50%) of the graft degrades within one year of implantation.

As used herein, unless otherwise stated, the term “physiological conditions” refers to the range of chemical (e.g., pH, ionic strength, and the like) and biochemical conditions (e.g., enzyme concentrations and the like) likely to be encountered in the intracellular and extracellular fluids of tissues. For most tissues, the physiological pH ranges from about 7.0 to 7.4.

As used herein, unless otherwise stated, the term “scaffold” refers to a structural support facilitating cell infiltration and attachment in order to guide vessel growth.

As used herein, the term “subject” refers to living multi-cellular vertebrate organisms, a category that includes human and non-human mammals (such as, for example, patients, laboratory or veterinary subjects, and the like). In an example, a subject is a human. In another additional example, a subject is in need of an implant for damaged or defective artery.

As used herein, unless otherwise stated, a “vascular graft” is a term used to refer to a tubular member which acts as an artificial vessel.

As used herein, unless otherwise stated, a “small molecule” is a term used to refer to molecules, whether naturally-occurring or artificially created (e.g., via chemical synthesis), that have a relatively low molecular weight. Typically, small molecules are monomeric and have a molecular weight of less than about 1500 g/mol. Small molecules may be biologically active in that they produce a local or systemic effect in animals, such as, for example, mammals (e.g., humans) and the like. In certain examples, a small molecule is a drug. It may be desirable a drug is one that has already been deemed safe and effective for use by the appropriate governmental agency or body. In various examples, drugs for human use listed by the FDA under 21 C.F.R. §§330.5, 331 through 361, and 440 through 460; drugs for veterinary use listed by the FDA under 21 C.F.R. §§500 through 589, and all these drugs considered acceptable for use in accordance with the present disclosure.

As used herein, unless otherwise stated, the term “tissue” refers to a collection of similar cells combined to perform a specific function, and may include any extracellular matrix surrounding the cells.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

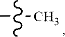

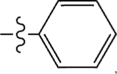

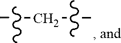

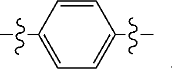

The present disclosure describes poly(glycerol sebacate) (PGS) polymers and polymeric materials. The present disclosure also provides compositions comprising the PGS polymers and polymeric materials and uses of the PGS polymers, polymeric materials, and compositions.

In the present disclosure, strategies were developed to make functionalized poly(glycerol sebacate)s, such as, for example, palmitate-functionalized poly(glycerol sebacate) (PPGS) or benzoate-functionalized poly(glycerol sebacate) (BPGS). Without intending to be bound by any particular theory, it is considered functionalized poly(glycerol sebacate)s may mediate one or more or all of the polymer hydrophobicity, crystallinity, microstructures and thermal properties. Changes of these intrinsic properties can impart tunable degradation profiles and mechanical properties to the resultant elastomers depending on the pendant group content. For example, the elastic modulus is tuned from initially 838 ± 55 kPa for the PGS to 333 ± 21 kPa for the PPGS with 16 mol.% of palmitate pendants. These elastomers therefore become softer to undergo reversible elastic deformations for at least 1000 cycles within 20% strain without failure and show enhanced elasticity when the palmitate pendants reach 9 to 16 mol.%. Because the materials are made from endogenous molecules, they possess good cytocompatibility similar to the PGS control. An application of the instant materials is to engineer synthetic vascular grafts for small artery remodeling. A slower degradation and more compliant elastic performance result in the neo-arteries closer to the native arteries in a rat carotid artery interposition model. Although certain materials were designed specifically for small arteries, it is expected that materials of the present disclosure are useful for other soft tissues as well.

Without intending to be bound by any particular theory, it is considered PGS polymers of the instant disclosure, which may have a slower degradation and/or more compliant properties and/or enhanced elasticity relative to a similar unfunctionalized PGS polymer, may exhibit improved remodeling efficiency for the arterial regeneration. In various examples, the PGS polymers of the present disclosure (1) adjust the hydrophobicity of the polymer and thus the degradation of the polymer and/or (2) enhance the elasticity for reversible mechanical deformations and/or (3) adjust the elastic modulus to match the biomechanical properties of soft tissues including the small arteries and/or (4) remain good biocompatibility and bioresorbability for tissue grafts. In various examples, in vivo studies revealed that synthetic vascular grafts made from PPGS with 16 mol.% palmitate pendants demonstrated an improved artery remodeling efficiency.

In an aspect, the present disclosure provides PGS polymers and polymeric materials. The PGS polymers and polymeric materials comprise pendant aliphatic carboxylate groups and/or pendant aryl carboxylate groups. Each pendant aliphatic carboxylate group and/or pendant aryl carboxylate group is covalently bound to the glycerol group of the glycerol sebacate backbone of the polymer. Reference herein to aliphatic carboxylate groups, unless otherwise stated, is intended to include aliphatic carboxylate groups comprising fully or partially protonated and fully or partially deprotonated acid groups. A PGS polymer may be referred to as a functionalized poly(glycerol sebacate) polymer. A PGS polymer or polymeric material may be prepolymer. A PGS polymer may be an elastomer. Non-limiting examples of PGS polymers and polymeric materials are described herein.

The pendant aliphatic carboxylate groups may be saturated aliphatic carboxylate groups, which may be linear saturated aliphatic carboxylate groups, or unsaturated aliphatic carboxylate groups, which may be linear unsaturated aliphatic carboxylate groups. The pendant saturated aliphatic carboxylate groups may be fatty acid carboxylate groups. The pendant saturated aliphatic carboxylate groups, which may be pendant fatty acid carboxylate groups, may be formed or derived from (or correspond to) saturated aliphatic carboxylic acids, which may be naturally-occurring fatty acids.

A PGS polymer can be of various sizes. In various examples, a PGS polymer is an oligomer. In various examples, a PGS polymers has degree of polymerization of 10 or greater.

A PGS polymer can have various end groups. A PGS polymer or PGS group of a polymeric material may comprise end groups independently chosen from hydrogen group, alkyl groups (e.g., a methyl group and the like), sebacate group, hydroxyl group, ester groups, amide groups, and the like, and combinations thereof.

A PGS polymer can have various conformations. A PGS polymer may be a crosslinked random coil.

A PGS polymer may have various morphologies. In various examples, a poly(glycerol sebacate) polymer is semicrystalline. In various examples, a PGS comprises one or more crystalline domain(s).

In various examples, a PGS polymer comprises (or has) the following structure:

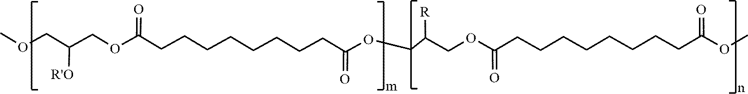

where R is a pendant group independently chosen from aliphatic carboxylate groups (e.g., fatty acid carboxylate groups) and aryl carboxylate groups, R′ is a hydrogen group, m is 0 to 99, including all integer numbers and ranges therebetween, and n is 1 to 100, including all integer numbers and ranges therebetween. m an n are each molar % values. In various examples, 1 to 100% of the glycerol groups, including all 0.1% values and ranges therebetween, of the PGS polymer have a pendant aliphatic carboxylate group and/or pendant aryl carboxylate group and/or 2 to 20%, 10 to 20%, 15 to 30 %, or 50 to 70% of the glycerol groups of the poly(glycerol sebacate) polymer have a pendant aliphatic carboxylate group and/or a pendant aryl carboxylate group.

A PGS polymer can comprise various pendant aliphatic carboxylic acid/carboxylate groups. A PGS polymer may have a combination of pendant aliphatic carboxylic acid/carboxylate groups. One or more pendant aliphatic carboxylic acid/carboxylate group(s) may have a different number of carbons that one or more of the other pendant carboxylic acid/carboxylate group(s). A pendant aliphatic carboxylate group may comprise (or be) a C₃ to C₄₀ aliphatic carboxylate group, including all integer numbers of carbons and ranges therebetween. The pendant aliphatic groups may be unsaturated fatty acid groups, saturated fatty acid groups, or a combination thereof. Non-limiting examples of pendant aliphatic carboxylate groups include butyrate groups, palmitate groups, stearate groups, oleate groups, substituted derivatives thereof, deprotonated analogs thereof, and the like, and combinations thereof. Any of the aforementioned groups may be covalently bound to the polymer backbone via a carbon of the aliphatic chain of the group.

An individual pendant aliphatic carboxylate group may be formed or derived from (or a group corresponding to) an unsaturated fatty acid. Non-limiting examples, of unsaturated fatty acids include, mono-unsaturated fatty acids (e.g., crotonic acid, myristoleic acid, palmitoleic acid, sapienic acid, oleic acid, elaidic acid, vaccenic acid, gadoleic acid, eicosenoic acid, erucic acid, nervonic acid, and the like), di-unsaturated fatty acids (such as, for example, linoleic acid, eicosadienoic acid, docosadienoic acid, and the like), tri-unsaturated fatty acids (such as, for example, a-linolenic acid, y-linolenic acid, pinolenic acid, α-eleostearic acid, β-eleostearic acid, mead acid, dihomo-γ-linolenic acid, eicosatrienoic acid, and the like), tetra-unsaturated fatty acids (such as, for example, stearidonic acid, arachidonic acid, eicosatetraenoic acid, adrenic acid, and the like), penta-unsaturated fatty acids (such as, for example, bosseopentaenoic acid, eicosapentaenoic acid, ozubondo acid, sardine acid, tetracosanolpentaenoic acid, and the like), hexa-unsaturated fatty acids (e.g., cervonic acid, herring acid, and the like)), and the like, groups formed therefrom, and combinations thereof.

Non-limiting examples, of unsaturated fatty acids groups include, mono-unsaturated fatty acid groups (e.g., crotonic acid groups, myristoleic acid groups, palmitoleic acid groups, sapienic acid groups, oleic acid groups, elaidic acid groups, vaccenic acid groups, gadoleic acid groups, eicosenoic acid groups, erucic acid groups, nervonic acid groups, and the like), di-unsaturated fatty acid groups (such as, for example, linoleic acid groups, eicosadienoic acid groups, docosadienoic acid groups, and the like), tri-unsaturated fatty acid groups (such as, for example, a-linolenic acid groups, y-linolenic acid groups, pinolenic acid groups, α-eleostearic acid groups, β-eleostearic acid groups, mead acid groups, dihomo-γ-linolenic acid groups, eicosatrienoic acid groups, and the like), tetra-unsaturated fatty acid groups (such as, for example, stearidonic acid groups, arachidonic acid groups, eicosatetraenoic acid groups, adrenic acid groups, and the like), penta-unsaturated fatty acid groups (such as, for example, bosseopentaenoic acid groups, eicosapentaenoic acid groups, ozubondo acid groups, sardine acid groups, tetracosanolpentaenoic acid groups, and the like), hexa-unsaturated fatty acid groups (e.g., cervonic acid groups, herring acid groups, and the like)), substituted derivatives thereof, deprotonated analogs thereof, and the like, and combinations thereof. Any of the aforementioned groups may be covalently bound to the polymer backbone via a carbon (which may be a terminal carbon) of the aliphatic chain of the group.

An individual pendant aliphatic carboxylate group may be formed or derived from (or a group corresponding to) a saturated fatty acid. In various examples, a fatty acid group is —CHₙ(CH₂)ₙCOOH, wherein n = 1-38, including all integer numbers and ranges therebetween. Non-limiting examples of saturated fatty acids include propionic acid, butyric acid, valeric acid, caproic acid, enanthic acid, caprylic acid, pelargonic acid, capric acid, undecylic acid, lauric acid, tridecylic acid, myristic acid, pentadecylic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, psyllic acid, geddic acid, ceroplastic acid, hexatriacontylic acid, heptatriacontylic acid, octatriacontylic acid, nonatriacontylic acid, tetracontylic acid, and the like, groups formed therefrom, and combinations thereof.

Non-limiting examples of saturated fatty acid groups include propionic acid groups, butyric acid groups, valeric acid groups, caproic acid groups, enanthic acid groups, caprylic acid groups, pelargonic acid groups, capric acid groups, undecylic acid groups, lauric acid groups, tridecylic acid groups, myristic acid groups, pentadecylic acid groups, margaric acid groups, stearic acid groups, nonadecylic acid groups, arachidic acid groups, heneicosylic acid groups, behenic acid groups, tricosylic acid groups, lignoceric acid groups, pentacosylic acid groups, cerotic acid groups, carboceric acid groups, montanic acid groups, nonacosylic acid groups, melissic acid groups, hentriacontylic acid groups, psyllic acid groups, geddic acid groups, ceroplastic acid groups, hexatriacontylic acid groups, heptatriacontylic acid groups, octatriacontylic acid groups, nonatriacontylic acid groups, tetracontylic acid groups, substituted derivatives thereof, deprotonated analogs thereof, and the like, and combinations thereof. Any of the aforementioned groups may be covalently bound to the polymer backbone via a carbon of the aliphatic chain of the group.

An individual aliphatic carboxylate group may be formed or derived from (or a group corresponding to) an aromatic carboxylic acid. The aryl group of an aryl carboxylate group may be a fused ring aryl group (e.g., naphthyl groups and the like) or a biaryl group (e.g., biphenyl groups and the like). Non-limiting examples of aromatic carboxylic acids include benzoic acid, 2-sulfonate-benzoic acid, 4-trifluoromethyl-benzoic acid, 4-dimethylaminobenzoic acid, 2,3,4,5,6-pentafluorobenzoic acid, and the like. In various examples, a pendant aryl carboxylate group is a C₆ to C₁₂ aryl carboxylate group. Non-limiting examples of aryl carboxylate and substituted aryl carboxylate groups include benzoate groups, 2-sulfonate-benzoate groups, 4-trifluoromethyl-benzoate groups, 4-dimethylaminobenzoate groups, 2,3,4,5,6-pentafluorobenzoate groups, substituted derivatives thereof, deprotonated analogs thereof, and the like, and combinations thereof. Any of the aforementioned groups may be covalently bound to the polymer backbone via a carbon of the aliphatic chain of the group.

A PGS polymer can have desirable properties. In various examples, the polymer exhibits one or more of the following desirable property(ies):
hydrophobicity (e.g., water contact angle of 66 to 85 degrees for a PGS polymer with a palmitate content from 0 to 16 mol.%. It is expected that the contact angle will increase with greater palmitate content,
elasticity (e.g., an elastic modulus of 840 to 330 kPa for a PGS polymer with a palmitate content from 0 to 16 mol.%),
biocompatibility,
biodegradability,
bioresorbabilty.

In various examples, a PGS polymer exhibits one or more or all of the following:
Simultaneously adjust the hydrophobicity, the degradation and elastic modulus for soft tissue engineering applications through changing the contents of pendants on the PGS.
Pendant groups make the elastomer softer with enhanced elasticity for reversible deformations.
The materials comprise endogenous molecules (e.g., fatty acids, glycerol, and sebacate). The PGS materials exhibit good biocompatibility and bioresorbability (e.g., similar to unfunctionalized PGS), which may be desirable for tissue grafts.

A PGS polymer or polymeric material may be at least partially crosslinked. In various examples, a PPG polymer or polymeric material comprises a plurality of intrachain and/or interchain crosslinks. A PGS polymer can be crosslinked by methods known in the art. In various examples, a PGS polymer is thermally crosslinked. The thermal crosslinking may be carried out under reduced pressure. In various examples, a plurality of the pendant aliphatic carboxylate groups and/or pendant aryl carboxylate groups of a PGS polymer or a polymeric material are each covalently bound to two glycerol groups of the glycerol sebacate backbone, thereby forming a crosslinked elastomeric poly(glycerol sebacate) or polymeric material, which may be a material or a tissue graft. A PGS polymer may be pre-polymer that is further crosslinked to form a polymer network. In various examples, a network polymer comprises PGS polymer domains.

A polymeric material comprises a plurality of glycerol sebacate groups. At least a portion of the individual glycerol sebacate groups have a pendant aliphatic carboxylate group and/or pendant aryl carboxylate group covalently bound to the glycerol group of the glycerol sebacate group. The pendant aliphatic carboxylate groups and pendant aryl carboxylate groups are as defined herein. In various examples, 1 to 100% of the glycerol groups, including all 0.1% values and ranges therebetween, of the glycerol sebacate groups have a pendant aliphatic carboxylate group and/or pendant aryl carboxylate group. In various other examples, 2 to 20%, 10 to 20%, 15 to 30%, or 50 to 70% of the glycerol groups of the glycerol sebacate groups have a pendant aliphatic carboxylate group and/or a pendant aryl carboxylate group. A polymeric material may be semicrystalline. A polymeric material may have various morphologies.

In various examples, a polymeric material comprises the following structure:

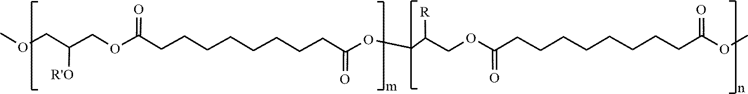

where R is a pendant group independently chosen from aliphatic carboxylate groups (e.g., fatty acid carboxylate groups) and aryl carboxylate groups, R′ is independently chosen from hydrogen group, sebacate group, oligo(glycerol sebacate) groups, and poly(glycerol sebacate) groups (where at least a portion of R′ group(s) are sebacate groups, oligo(glycerol sebacate) groups, poly(glycerol sebacate) groups, or a combination thereof, or each R′ group(s) is not a hydrogen group), m is 0 to 99, including all integer numbers and ranges therebetween, and n is 1 to 100, including all integer numbers and ranges therebetween. m an n are each molar % values. In various examples, 1 to 100% of the glycerol groups, including all 0.1% values and ranges therebetween, of the poly(glycerol sebacate) polymer have a pendant aliphatic carboxylate group and/or pendant aryl carboxylate group and/or 2 to 20%, 10 to 20%, 15 to 30%, or 50 to 70% of the glycerol groups of the poly(glycerol sebacate) polymer have a pendant aliphatic carboxylate group and/or a pendant aryl carboxylate group.

A polymeric material may be a copolymer. A copolymer may be a block copolymer. In various examples, a block copolymer comprises one or more poly(glycerol sebacate) block(s) formed from a plurality of the glycerol sebacate groups. A block copolymer may comprise one or more additional block(s) chosen from hydrophilic blocks, hydrophobic blocks, and the like, and combinations thereof. Non-limiting examples of hydrophilic blocks include polyethylene glycol (PEG) blocks, polypropylene glycol blocks, hyaluronan blocks, chitosan blocks, carbohydrate blocks, and the like, and combinations thereof. Non-limiting examples of hydrophobic blocks include polyethylene terephthalate (PET) blocks, poly(caprolactone) (PCL) blocks, polylactic acid (PLA) blocks, polyglycolic acid (PGA) blocks, and the like, and combinations thereof.

A PGS polymer or polymeric material may be made by methods described herein. In various examples, a PGS polymer or polymeric material is made by reacting a preformed, unfunctionalized PGS polymer or unfunctionalized poly(glycerol sebacate) polymer with one or more aliphatic carboxylic acid anhydride(s).

In an aspect, the present disclosure provides compositions. The compositions comprise one or more PGS polymer(s) and/or one or more polymeric material(s). Non-limiting examples of compositions are described herein.

PGS polymers and/or polymeric materials may be combined with other polymers in blends and adducts to manipulate the degradation and mechanical properties of the material. Practically any biocompatible polymers may be combined with functionalized PGS material. In various examples, the added polymer(s) is/are biodegradable. The added polymer(s) may be hydrolytically degradable or the like). Non-limiting examples of biodegradable polymers include natural polymers and their synthetic analogs, including polysaccharides, proteoglycans, glycosaminoglycans, collagen-GAG, collagen, fibrin, and other extracellular matrix components (such as, for example, elastin, fibronectin, vitronectin, laminin, and the like), and the like, and combinations thereof. Hydrolytically degradable polymers are known in the art. Non-limiting examples of hydrolytically degradable polymers include certain polyesters, polyanhydrides, polyorthoesters, polyphosphazenes, polyphosphoesters, and the like. Biodegradable polymers are known in the art. Non-limiting examples, of biodegradable polymers include certain polyhydroxyacids, polypropylfumerates, polycaprolactones, polyhydroxyalkanoates, poly(amide-enamines), polyamides, poly(amino acids), polyacetals, polyethers, biodegradable polycyanoacrylates, biodegradable polyurethanes and polysaccharides, and the like. For example, specific biodegradable polymers that may be used include but are not limited to, polylysine, poly(lactic acid) (PLA), poly(glycolic acid) (PGA), copolymers and mixtures of PLA and PGA, e.g., poly(lactide-co-glycolide) (PLG), poly(caprolactone) (PCL), poly(lactide-co-caprolactone) (PLC), and poly(glycolide-co-caprolactone) (PGC). The properties of these and other polymers and methods for preparing them are further described in the art. See, for example, U.S. Pat. Nos. 6,123,727; 5,804,178; 5,770,417; 5,736,372; 5,716,404 to Vacanti; 6,095,148; 5,837,752 to Shastri; 5,902,599 to Anseth; 5,696,175; 5,514,378; 5,512,600 to Mikos; 5,399,665 to Barrera; 5,019,379 to Domb; 5,010,167 to Ron; 4,806,621; 4,638,045 to Kohn; and 4,946,929 to d′Amore; see also Wang et al., J. Am. Chem. Soc. 123:9480, 2001; Lim et al., J. Am. Chem. Soc. 123:2460, 2001; Langer, Acc. Chem. Res. 33:94, 2000; Langer, J. Control Release 62:7, 1999; and Uhrich et al., Chem. Rev. 99:3181, 1999.

PGS polymers and/or polymeric materials may also be combined with non-biodegradable polymers. For example, polypyrrole, polyanilines, polythiophene, and derivatives thereof are useful electrically conductive polymers that can provide additional stimulation to seeded cells or neighboring tissue. Nonlimiting examples of non-biodegradable polymers include polystyrene, polyesters, non-biodegradable polyurethanes, polyureas, poly(ethylene vinyl acetate), polypropylene, polymethacrylate, polyethylene, polycarbonates, poly(ethylene oxide), and the like, and combinations thereof.

A composition may comprise (or be) a fiber or a plurality of fibers. The fiber(s) comprise one or more PGS polymer(s) and/or one or more polymeric material(s). A fiber may be formed by electrospinning (e.g., electrospun with gelatin). A fiber may be an electrospun fiber. A fiber or plurality of fibers may be used to form a material, such as, for example, a fabric.

Other fibers (e.g., fibers that do not comprise one or more PGS polymer(s) and/or one or more polymeric material(s)) and particles may be combined with the PGS polymer(s) and/or polymeric material(s). The other fibers and/or particles may modify the mechanical properties of the PGS polymer(s) and/or polymeric material(s). In various examples, fibers (such as, for example, collagen, PLGA, or the like, which may be embedded in the PGS polymer(s) and/or polymeric material(s) to stiffen the PGS polymer(s) and/or polymeric material(s) and/or particles (such as, for example, Bioglass™, calcium phosphate ceramics, or the like, are combined with the PGS polymer(s) and/or polymeric material(s).

A composition may comprise one or more other fibers (e.g., fibers that do not comprise one or more PGS polymer(s) and/or one or more polymeric material(s)). In various examples, a composition is a material comprising one or more fiber(s) and, optionally, one or more other fiber(s). A fiber may be a blend of one or more poly(glycerol sebacate)(s), one or more polymeric material(s), or a combination thereof, and one or more other polymer(s) and/or one or more other polymeric material(s). In various examples, a fiber (e.g., a blended fiber) is formed by electrospinning using a solution comprising one or more poly(glycerol sebacate)(s), one or more polymeric material(s), or a combination thereof, and optionally, one or more other polymer(s) and/or one or more other polymeric material(s). Non-limiting other polymer(s) and polymeric material(s) include polylactic acids (PLAs), polyglycolic acids (PGAs), PLGAs, poly(caprolactone)s (PCLs), polyethylene glycols (PEGs), polyethylene terephthalates (PETs), polypropylenes, polyethylenes, nylons, polystyrenes, and the like, and combinations thereof.

A material comprises (or is) one or more PGS polymer(s) and/or polymeric material(s). One or more of the PGS polymer(s) and/or polymeric material(s) may be at least partially crosslinked (or a crosslinked network). A material can have various shapes. In various examples, a material has a shape chosen from particles, tubes, spheres, strands, coiled strands, capillary networks, films, fibers, meshes, sheets, and the like.

A material may comprise a plurality of one or more fiber(s). A material may be a fabric. A fabric may be a woven fabric. A fabric may be a weave or braid of one or more fiber(s). A material may comprise one or more other fiber(s) (e.g., fibers that do not comprise one or more PGS polymer(s) and/or one or more polymeric material(s)). An other fiber may comprise a degradable polymer and/or a non-biodegradable fiber. Non-limiting other fibers include polylactic acid (PLA) fibers, polyethylene glycol (PEG) fibers, PLGA fibers, poly(lactide-co-caprolactone) (PLCL) fibers, polyglycolic acid (PGA) fibers, PLGA fibers, poly(caprolactone) (PCL) fibers, polyethylene terephthalate (PET) fibers, polypropylene fibers, polyethylene fibers, nylon fibers, polystyrene fibers, and the like, and combinations thereof.

The PGS polymers and/or polymeric materials, and optionally other materials, may be electrospun to form scaffolds of any desired shape, such as, for example sheets, tubes, meshes, pseudo 3-dimensional constructs. It is contemplated that the constructs may be of high porosity, low porosity, a combination of different porosity. In various examples, vascularized (micro-channeled) fibrous sheets, random meshes, aligned sheets, cylindrical tubes, or pseudo 3-dimensional constructs, such as, for example, shapes to mimic organs, are formed. In various other examples, complex shapes such as, for example, those mimicking organs are formed. Electrospinning with a sacrificial template can be used to create highly porous scaffolds to mimic ECM. It is contemplated that the constructs/scaffolds can be used to guide host tissue remodeling in many different tissues, for example, including any tissue that has progenitor cells. A biodegradable scaffold may be used to facilitate tissue regeneration in vivo by providing a structural frame for which tissue regeneration can occur. In various examples, a PGS polymer and/or polymeric material is electrospun such that it allows and facilitates the infiltration of host cells, for example, including progenitor cells. In various examples, a composition is such that it allows and facilitates host remodeling of the polymer, so that eventually the polymeric structure is replaced by the desirable host tissue.

A composition may be a tissue graft. PGS polymer(s), polymeric material(s), compositions, or a combination thereof (or fibers or materials comprising PGS polymer(s) and/or polymeric material(s)) may be used to tissue engineer, for example, epithelial, connective, nerve, muscle, organ, and other tissues. Non-limiting examples of tissues include artery, ligament, skin, tendon, kidney, nerve, liver, pancreas, bladder, and the like. PGS polymers, polymeric materials, compositions, or a combination thereof may be used to tissue engineer regenerating tissues that are subject to repeated tensile, hydrostatic, or other stresses. Examples of such tissues include, but are not limited to, lung, blood vessels, heart valve, bladder, cartilage, muscle, and the like.

A tissue graft may be a porous matrix or porous matrices that create 3-dimensional scaffolds for cell ingrowth from the host or cell seeding for tissue engineered organ approaches. A tissue graft may be a cell-free scaffold or graft, such as, for example, a cell-free vascular graft, in which the graft does not include any living cells, such as, for example, smooth muscle cells, endothelial cells, stem cells, progenitor cells, or the like, or a combination thereof. A tissue graft may be a fibrous network. In various examples, a tissue graft comprises one or more PGS polymers, one or more polymeric material(s), or a combination thereof, and/or a fiber or material comprising one or more poly(glycerol sebacate)(s), one or more polymeric material(s), or a combination thereof.

The shape of a PGS polymer, polymeric material, or composition may also be manipulated for specific tissue engineering applications. A tissue graft may have various shapes. Non-limiting examples of shapes include particles, tubes, spheres, strands, coiled strands, films, sheets, fibers, meshes, pseudo 3-dimensional constructs, and the like. In various examples, a tissue graft is a capillary network. Microfabrication may be used to form a capillary networks.

These shapes may be exploited to engineer a wide variety of tissues. For example, PGS polymer(s), polymeric material(s), or composition(s) is/are fabricated into a tube to facilitate nerve regeneration. In another example, a PGS polymer, polymeric material, or composition is be used to fabricate the tissue structures of liver. For example, it may be formed into a network of tubes that mimic a blood vessel and capillary network which may be connected to a nutrient supply to carry nutrients to the developing tissue. PGS polymer(s), polymeric material(s), or composition(s) may also be seeded with a variety of other cells, such as, for example, tenocytes, fibroblasts, ligament cells, endothelial cells, epithelial cells, muscle cells, nerve cells, kidney cells, bladder cells, intestinal cells, chondrocytes, bone-forming cells, stem cells such as, for example human embryonic stem cells or mesenchymal stem cells, and others, and combinations thereof.

In various examples, a salt leaching technique may be used to make tubes or disk to give adapted shape for the use. Using a salt leaching technique, highly porous scaffolds, with a range of porosity may obtained depending on the salt crystal size, packing skills, or the like.

In various examples, a scaffold or graft comprises uniformly distributed pores. In various examples, a scaffold or graft comprises non-uniformly distributed pores. In various examples, a scaffold or graft does not include any pores. In various examples, a scaffold or graft comprises at least 75% pore interconnectivity, such as, for example about 80% to about 90%, about 90% to about 98%, including 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% interconnectivity.

A tissue graft may be for any soft tissues (such as, for example, muscle, skin, ligaments, internal organs, and the like). A tissue graft may be a soft tissue graft. Non-limiting examples of soft tissue grafts include muscle grafts, skin grafts, ligament grafts, internal organ grafts, and the like). A tissue graft may be a vascular graft. A vascular graft may be arterial graft, which may be a small artery graft. The various dimensions of a disclosed scaffold or tissue graft may vary according to the desired use. A small artery graft may have a lumen diameter of 6 mm or less.

A tissue graph may comprise a polymer component (which is not a PGS component, such as, for example, PETE) that is generally considered to be non-biodegradable. In various examples, a tissue graft comprises thin fibers and an open porous structure that may degrade over 3 years.

A graft may comprise one or more other material(s) (e.g., non-PGS and/or non-polymeric materials). In the case of materials comprising one or more other fiber(s), one or more of the other fiber(s) degrades (e.g., biodegrades in an individual) forming a scaffold comprising the remaining fibers.

To further control or regulate polymer interaction with cells, biomolecules, small molecules, or bioactive agents may be coupled to the hydroxyl groups or integrated into the polymer backbone (Barrera, D., et al., Synthesis and RGD Peptide Modification of a New Biodegradable Copolymer: Poly(lactic acid-co-lysine). J. Am. Chem. Soc. 115: 11010-11, 1993; West, J. L., et al., Polymeric Biomaterials with Degradation Sites for Proteases Involved in Cell Migration. Macromolecules 32: 241-244, 1999; Mann, B. K., Smooth Muscle Cell Growth in Photopolymerized Hydrogels with Cell Adhesive and Proteolytically Degradable Domains: Synthetic ECM Analogs for Tissue Engineering. Biomaterials 22, 3045-3051; 2001).

A PGS polymer or polymeric material (which may be a fiber, a material, or a tissue graft) may comprise a biomolecule, a small molecule, a bioactive molecule, or any combination thereof. Non-limiting examples of biomolecules include growth factors, cell adhesion sequences, polynucleotides, polysaccharide, polypeptide, extracellular matrix components or the like, and combinations thereof. A biomolecule, a small molecule, or a bioactive molecule may be linked to the polymer backbone through one more covalent bond(s), one or more hydrogen bond(s), an electrostatic interaction, a hydrophobic interaction, and a van der Waals interaction, or the like, or a combination thereof.

It may be desirable that the poly(glycerol sebacate) component(s) in a tissue graft is/are biodegraded by host cells in 2 weeks to 3 months. A tissue graph may comprise a polymer component (which is not a PGS component, such as, for example, for example, PETE) that is generally considered to be non-biodegradable. In various examples, a tissue graft comprises thin fibers and an open porous structure that may degrade over 3 years.

In various examples, at least 50%, such as, for example about 55% to about 70%, about 80% to about 90%, about 90% to about 98%, including 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% of a scaffold or tissue graft, which may be a vascular graft, degrades within one year, such as, for example within 1 to 10 months, including within 1 month, 2 months, 3 months, 4 months, 5 months, 6 months, 7 months, 8 months, 9 months, 10 months, 11 months, or 12 months of implantation. Degradation of the graft permits controlled and/or sustained release of the pendant carboxylic acid (e.g., palmitic acid).

PGS polymers, polymeric materials, and compositions may also be used for drug release applications, especially in applications where the matrix retaining the drug needs to be flexible. PGS polymers, polymeric materials, and compositions may also be used for wound care, such as, for example, for wounds that are hard to close or that fail to heal properly through normal physiologic mechanisms. For example, diabetics often get skin injuries (“diabetic ulcers”), especially in the lower extremities, that take a long time to heal or fail to heal properly due to poor circulation.

In various examples, PGS polymers, polymeric materials, and compositions comprise (e.g., are impregnated, coated on a surface of a composition (e.g., fiber, material, or the like, a scaffold, a graft, or the like with one or more, such as, for example two, three, four, five etc. suitable pharmaceutical agent(s). Suitable pharmaceutical agents may be organic or inorganic and may be in a solid, semisolid, liquid, or gas phase. A pharmaceutical agent may be a molecule. Molecules may be present in combinations or mixtures with other molecules, and may be in solution, suspension, or any other form. Examples of classes of pharmaceutical agents, which may be molecules, that may be used include, but are not limited to, human or veterinary therapeutics, cosmetics, nutraceuticals, agriculturals such as, for example, herbicides, pesticides and fertilizers, vitamins, salts, electrolytes, amino acids, peptides, polypeptides, proteins, carbohydrates, lipids, nucleic acids, glycoproteins, lipoproteins, glycolipids, glycosaminoglycans, proteoglycans, growth factors, hormones, neurotransmitters, pheromones, chalones, prostaglandins, immunoglobulins, monokines and other cytokines, humectants, metals, gases, minerals, plasticizers, ions, electrically and magnetically reactive materials, light sensitive materials, anti-oxidants, molecules that may be metabolized as a source of cellular energy, antigens, and any molecules that can cause a cellular or physiological response, and the like. Any combination of pharmaceutical agents can be used, as well as agonists or antagonists of these molecules.

Pharmaceutical agents include any therapeutic molecule including, without limitation, any pharmaceutical substance or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfiram and disulfiram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present disclosure.

In various examples, the inner luminal surface of a biodegradable scaffold is coated partially or completely with a thromboresistant agent, such as, for example heparin and/or other compounds known to one of skill in the art to have similar anti-coagulant properties as heparin, to prevent, inhibit or reduce clotting within the inner lumen of the vascular graft.

The following Statements describe examples of PGS polymers, materials, fibers, and tissue grafts of the present disclosure:

Statement 1. A poly(glycerol sebacate) (PGS) polymer, which may be referred to as a functionalized poly(glycerol sebacate) polymer, comprising pendant aliphatic carboxylate groups and/or pendant aryl carboxylate groups, each pendant aliphatic carboxylate group and/or pendant aryl carboxylate group is covalently bound to the glycerol group of the glycerol sebacate backbone of the polymer, which may be at least partially crosslinked. The pendant aliphatic carboxylate groups may be saturated aliphatic carboxylate groups, which may be linear saturated aliphatic carboxylate groups, or unsaturated aliphatic carboxylate groups, which may be linear unsaturated aliphatic carboxylate groups. The pendant saturated aliphatic carboxylate groups may be fatty acid carboxylate groups. The pendant saturated aliphatic carboxylate groups, which may be pendant fatty acid carboxylate groups, may be formed from saturated aliphatic carboxylic acids, which may be naturally occurring fatty acids. A PGS polymer may be an oligomer or have a degree of polymerization of 10 or greater. A PGS polymer may have end groups independently chosen from hydrogen group, alkyl groups (e.g., a methyl group), sebacate group, hydroxyl group, ester groups, amide groups, and the like. A PGS polymer may be pre-polymer that may be further crosslinked to form a polymer network. A PGS polymer may be an elastomer. A PGS polymer may be a crosslinked random coil. The poly(glycerol sebacate) polymer may be semicrystalline. Statement 2. A poly(glycerol sebacate) polymer according to Statement 1, wherein in the poly(glycerol sebacate) polymer has the following structure:

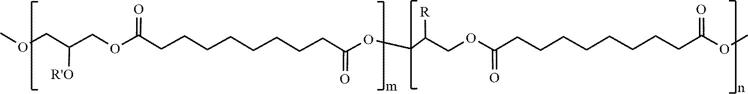

where R is a pendant group independently chosen from aliphatic carboxylate groups (e.g., fatty acid carboxylate groups) and aryl carboxylate groups, R′ is independently chosen from hydrogen group, sebacate, oligo(glycerol sebacate), and poly(glycerol sebacate), m is 0 to 99, and n is 1 to 100, where m an n are molar % values.

Statement 3. A poly(glycerol sebacate) polymer according to Statement 1 or 2, wherein 1 to 100% of the glycerol groups, including all 0.1% values and ranges therebetween, of the poly(glycerol sebacate) polymer have a pendant aliphatic carboxylate group and/or pendant aryl carboxylate group. In various examples, 2 to 20%, 10 to 20%, 15 to 30%, or 50 to 70% of the glycerol groups of the poly(glycerol sebacate) polymer have a pendant aliphatic carboxylate group and/or a pendant aryl carboxylate group.

Statement 4. A poly(glycerol sebacate) polymer according to any one of the preceding Statements, wherein the pendant aliphatic carboxylate groups are independently chosen from C₃ to C₄₀ aliphatic carboxylate groups, including all integer numbers of carbons and ranges therebetween. Non-limiting examples of aliphatic carboxylate groups include butyrate groups, palmitate groups, stearate groups, oleate groups, substituted analogs thereof, and the like, and combinations thereof. An individual aliphatic carboxylate group may be formed from (or a group corresponding to) an unsaturated fatty acid (such as, for example, mono-unsaturated fatty acids (e.g., crotonic acid, myristoleic acid, palmitoleic acid, sapienic acid, oleic acid, elaidic acid, vaccenic acid, gadoleic acid, eicosenoic acid, erucic acid, nervonic acid, and the like), di-unsaturated fatty acids (such as, for example, linoleic acid, eicosadienoic acid, docosadienoic acid, and the like), tri-unsaturated fatty acids (such as, for example, a-linolenic acid, y-linolenic acid, pinolenic acid, α-eleostearic acid, β-eleostearic acid, mead acid, dihomo-γ-linolenic acid, eicosatrienoic acid, and the like), tetra-unsaturated fatty acids (such as, for example, stearidonic acid, arachidonic acid, eicosatetraenoic acid, adrenic acid, and the like), penta-unsaturated fatty acids (such as, for example, bosseopentaenoic acid, eicosapentaenoic acid, ozubondo acid, sardine acid, tetracosanolpentaenoic acid, and the like), hexa-unsaturated fatty acids (e.g., cervonic acid, herring acid, and the like)), and the like. An individual aliphatic carboxylate group may be formed from (or a group corresponding to) a saturated fatty acid (e.g., CH₃(CH₂)ₙCOOH, wherein n = 1-38). Non-limiting examples of saturated fatty acids include propionic acid, butyric acid, valeric acid, caproic acid, enanthic acid, caprylic acid, pelargonic acid, capric acid, undecylic acid, lauric acid, tridecylic acid, myristic acid, pentadecylic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, psyllic acid, geddic acid, ceroplastic acid, hexatriacontylic acid, heptatriacontylic acid, octatriacontylic acid, nonatriacontylic acid, tetracontylic acid, and the like. An individual aliphatic carboxylate group may be formed from (or a group corresponding to) an aromatic acid benzoic acid, 2-sulfonate-benzoic acid, 4-trifluoromethyl-benzoic acid, 4-dimethylaminobenzoic acid, 2,3,4,5,6-pentafluorobenzoic acid, and the like.

Statement 5. A poly(glycerol sebacate) polymer according to any one of the preceding Statements, wherein the pendant aryl carboxylate groups are independently chosen from C₆ to C₁₂ aryl carboxylate groups. The aryl group of an aryl carboxylate group may be a fused ring aryl group (e.g., naphthyl groups and the like) or a biaryl group (e.g., biphenyl groups and the like). Non-limiting examples of aryl carboxylate and substituted aryl carboxylate groups include benzoate groups, 2-sulfonate-benzoate, 4-trifluoromethyl-benzoate, 4-dimethylaminobenzoate, 2,3,4,5,6-pentafluorobenzoate, substituted analogs thereof, and the like, and combinations thereof.

Statement 6. A polymeric material, which may be at least partially crosslinked, comprising a plurality of glycerol sebacate groups, wherein at least a portion of the individual glycerol sebacate groups have a pendant aliphatic carboxylate group and/or pendant aryl carboxylate group covalently bound to the glycerol group of the glycerol sebacate group. The pendant aliphatic carboxylate groups and pendant aryl carboxylate groups are as defined herein. In various examples, 1 to 100% of the glycerol groups, including all 0.1% values and ranges therebetween, of the glycerol sebacate groups have a pendant aliphatic carboxylate group and/or pendant aryl carboxylate group. In various other examples, 2 to 20%, 10 to 20%, 15 to 30%, or 50 to 70% of the glycerol groups of the glycerol sebacate groups have a pendant aliphatic carboxylate group and/or a pendant aryl carboxylate group. The polymeric material may be semicrystalline.

Statement 7. A polymeric material according to Statement 6, wherein the polymeric material is copolymer.

Statement 8. A polymeric material according to Statement 7, wherein the block copolymer comprises one or more poly(glycerol sebacate) block(s) formed from a plurality of the glycerol sebacate groups.

Statement 9. The polymeric material according to Statement 7 or 8, wherein the block copolymer comprise one or more additional block(s) chosen from hydrophilic blocks, hydrophobic blocks, and the like, and combinations thereof. Non-limiting examples of hydrophilic blocks include polyethylene glycol (PEG) blocks, polypropylene glycol blocks, hyaluronan blocks, chitosan blocks, carbohydrate blocks, and the like, and combinations thereof. Non-limiting examples of hydrophobic blocks include polyethylene terephthalate (PET) blocks, poly(caprolactone) (PCL) blocks, polylactic acid (PLA) blocks, polyglycolic acid (PGA) blocks, and the like, and combinations thereof.

Statement 10. A fiber comprising one or more poly(glycerol sebacate)(s) according to any one of Statements 1 to 5, one or more polymeric material(s) according to any one of Statements 6 to 9, or a combination thereof.

Statement 11. A fiber of Statement 10, wherein the fiber comprise one or more other polymer(s) and/or one or more other polymeric material(s). Non-limiting other polymer(s) and polymeric material(s) include polylactic acids (PLAs), polyglycolic acids (PGAs), PLGAs, poly(caprolactone)s (PCLs), polyethylene glycols (PEGs), polyethylene terephthalates (PETs), polypropylenes, polyethylenes, nylons, polystyrenes, and the like, and combinations thereof. The fiber may be a blend of one or more poly(glycerol sebacate)(s) of any one of Statements 1 to 5, one or more polymeric material(s) of any one of Statements 6 to 9, or a combination thereof, and one or more other polymer(s) and/or one or more other polymeric material(s).

Statement 12. A material comprising a plurality of one or more fiber(s) according to Statement 10 or 11.

Statement 13. A material of Statement 12, wherein the material is a fabric. The fabric may be a weave of one or more fiber(s) of Statement 10 or 11 and one or more other fiber(s). The fabric may be a weave or braid of one or more fiber(s) according to Statement 10 or 11. Statement 14. A material according to Statement 12 or 13, wherein the material comprises one or more other fiber(s). An other fiber may comprise a degradable polymer and/or a non-biodegradable fiber. Non-limiting other fibers include polylactic acid (PLA) fibers, polyethylene glycol (PEG) fibers, PLGA fibers, poly(lactide-co-caprolactone) (PLCL) fibers, polyglycolic acid (PGA) fibers, PLGA fibers, poly(caprolactone) (PCL) fibers, polyethylene terephthalate (PET) fibers, polypropylene fibers, polyethylene fibers, nylon fibers, polystyrene fibers, and the like, and combinations thereof.

Statement 15. A tissue graft comprising one or more poly(glycerol sebacate)(s) according to any one of Statements 1 to 5, one or more polymeric material(s) according to any one of Statements 6 to 9, or a combination thereof, and/or a fiber or material comprising one or more poly(glycerol sebacate)(s) according to any one of Statements 1 to 5, one or more polymeric material(s) according to any one of Statements 6 to 9, or a combination thereof (e.g., a fiber according to any one of Statements 10 or 11 or a material according to any one of Statements 12 to 14, or a combination thereof).

Statement 16. A tissue graft according to Statement 15, wherein, in the case of materials comprising one or more other fiber(s), one or more of the other fiber(s) degrades (e.g., biodegrades in an individual) forming a scaffold comprising the remaining fibers.

Statement 17. A tissue graft according to Statement 15 or 16, wherein the tissue graft is a vascular graft.

Statement 18. A tissue graft according to Statement 17, wherein the vascular graft is an arterial graft, which may be a small artery graft. It is desirable that the PGS component(s) in a tissue graft is/are biodegraded by host cells in 2 weeks to 3 months.

The following examples are presented to illustrate the present disclosure. The examples are not intended to be limiting in any matter.

### Example 1

This example describes PGS polymers (PGSs) of the present disclosure, methods of making PGS polymers, and characterization of PGS polymers.

This example focuses on material synthesis and characterization of palmitate-functionalized PGSs. Proton NMR, GPC and DSC analyses were used to examine the chemical structure, composition, molecular weight and thermal properties. SEM characterization was used to observe the microscopic structures formed in the elastomers relating to the palmitate pendants. Uniaxial tensile and cyclic tensile tests were performed to evaluate the mechanical properties and elasticity for reversible mechanical deformations. In addition, the material hydrophobicity relating to the palmitate contents were compared by water contact angle measurements; the effects on the degradation was evaluated in a basic solution and PBS (pH 7.4) respectively. Finally, cytocompatibility of the materials was examined on HUVECs by MTT and live/dead assays.

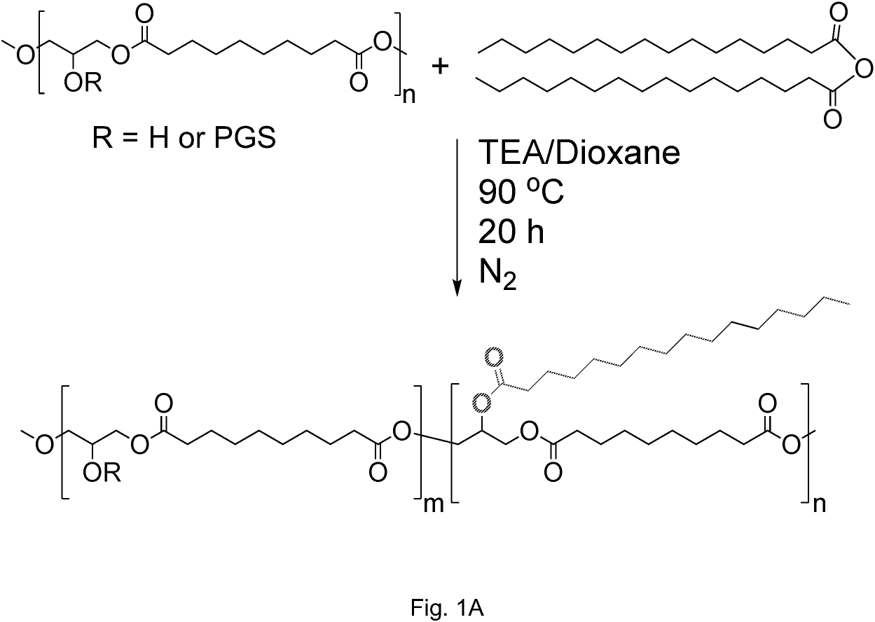

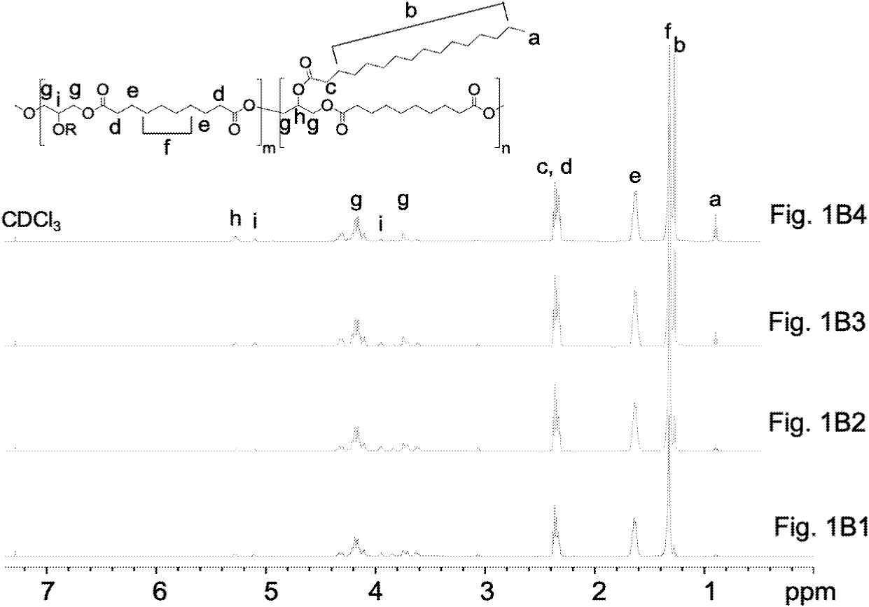

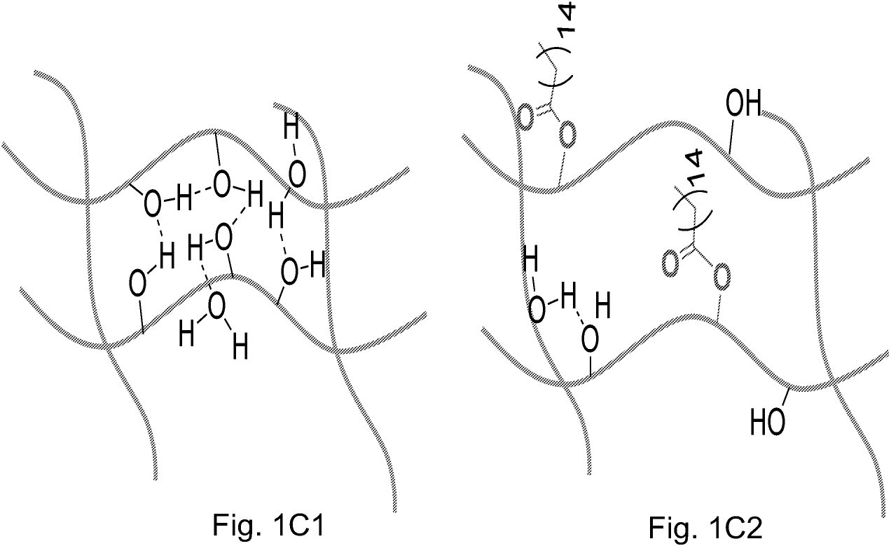

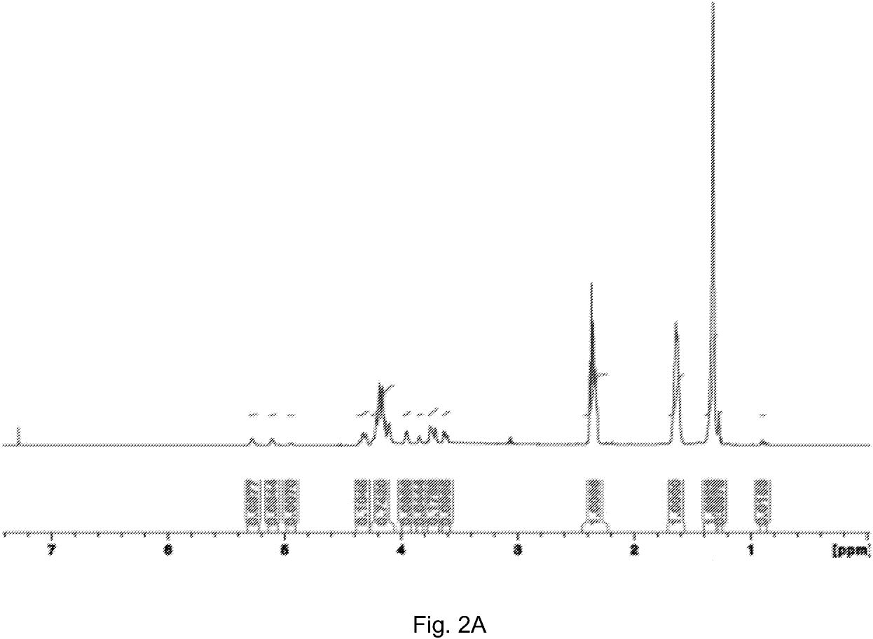

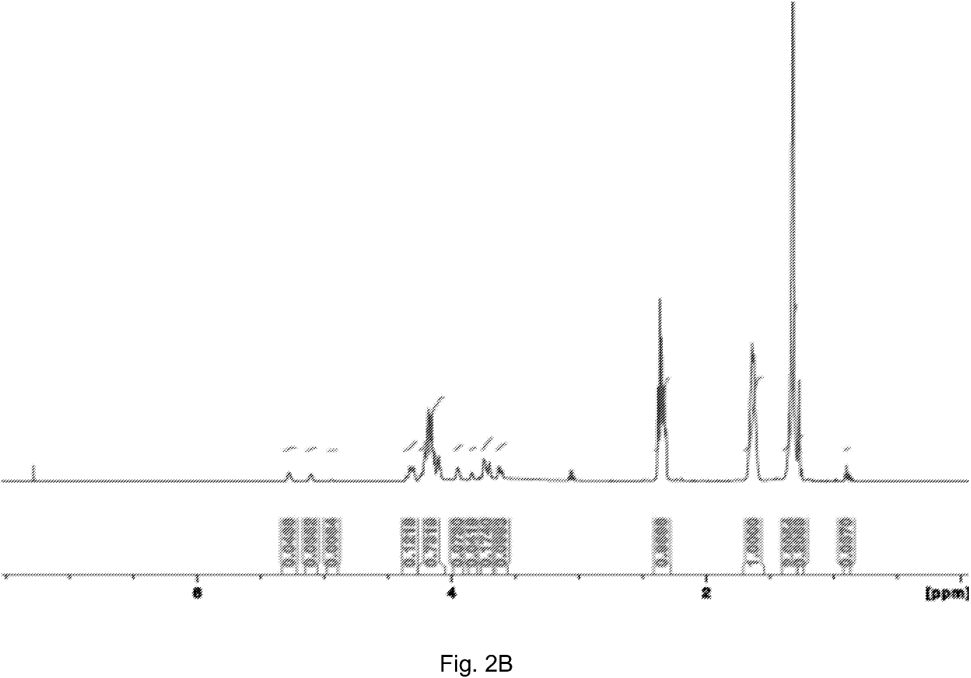

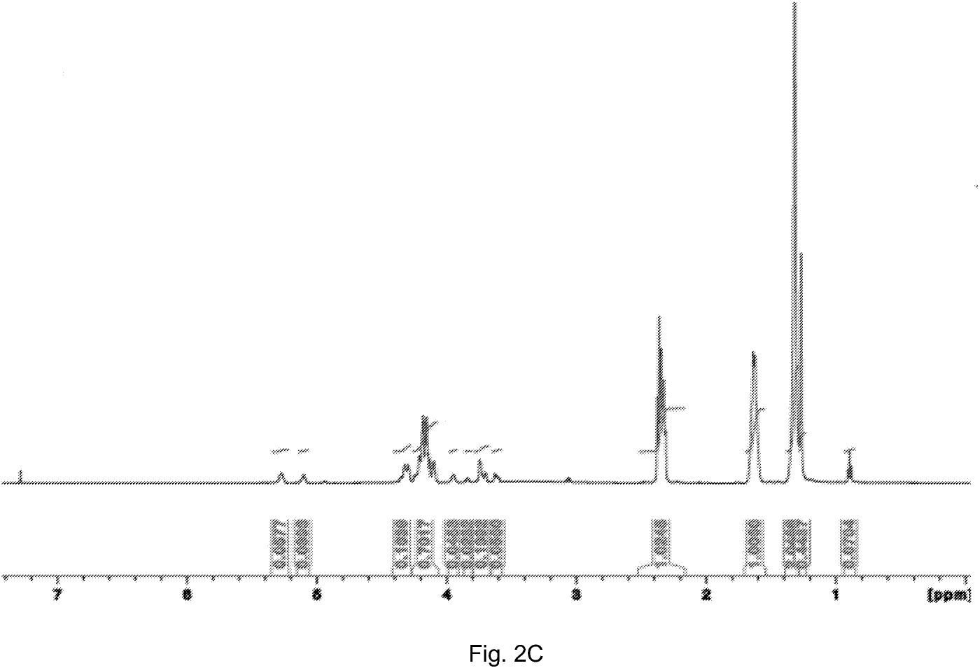

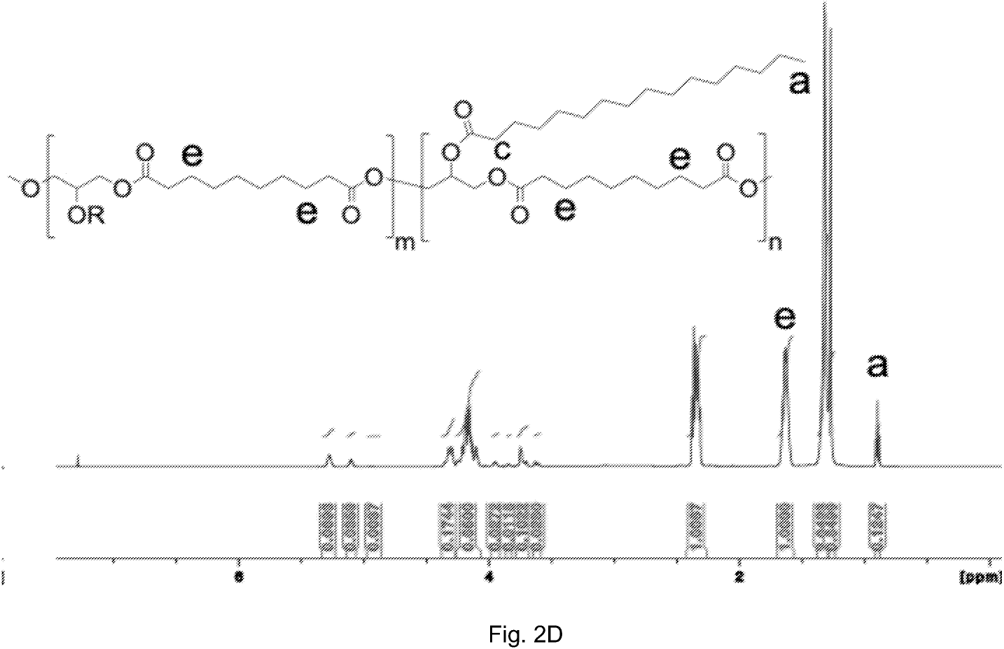

Synthesis and characterization of palmitate-functionalized poly(glycerol sebacate) (PPGS). The palmitate functionalities were introduced by reacting palmitic anhydrides with free hydroxyl groups on the PGS backbone in the presence of triethylamine catalyst (FIG. 1A). In this way, a series of PPGS pre-polymers were synthesized with different palmitate contents. These pre-polymers were made to investigate the effects of palmitate contents on the physicochemical, mechanical and biological properties. The PPGS pre-polymers were purified by repetitive dissolution in acetone and precipitation in hexanes to remove free palmitic acid byproducts and other chemical residues. The chemical structure and the actual palmitate contents in the pre-polymers were identified by proton NMR analysis. The chemical shifts were accordingly assigned to confirm the components of PGS and the immobilized palmitate pendants (FIG. 1B1-1B4). For example, the chemical shifts at 0.895 and 1.641 ppm were attributed to the protons of the methyl group (Hₐ) on the palmitate end and methylene groups (Hₑ) from the sebacate on the PPGS backbone. In addition, the palmitate pendants showed a chemical shift at 1.270 ppm for the methylene protons (H_{b}). The intensity was proportional to the palmitate contents and the signal was partially merged with the sebacate methylene protons (Hf) at 1.318 ppm (FIG. 1B1-1B4). The integral area ratio of Hₐ to Hₑ was used to determine the actual palmitate contents by the following equation:

$$\frac{3 {H}_{a} n}{4 {H}_{e} \left(n + m\right)} = \frac{A r e {a}_{{}^{{H}_{a}}}}{A r e {a}_{{H}_{e}}}$$

where (n+m) equals 100% and n represents the composition with the palmitate pendants, which was determined using the equation. According to the integral area ratios in FIGS. 2A-2D, the actual palmitate contents were calculated to be approximately 2, 5, 9 and 16 mol.%. The PPGS pre-polymers were therefore designated as PPGS-2, PPGS-5, PPGS-9 and PPGS-16. The proton NMR analysis indicated that 40 - 50% of the palmitic anhydrides were reacted with the hydroxyls on the PGS in these reactions. The yields of these pre-polymers ranged from 96% for the PPGS-2 to 74% for the PPGS-16 (FIG. 1B1-1B4).

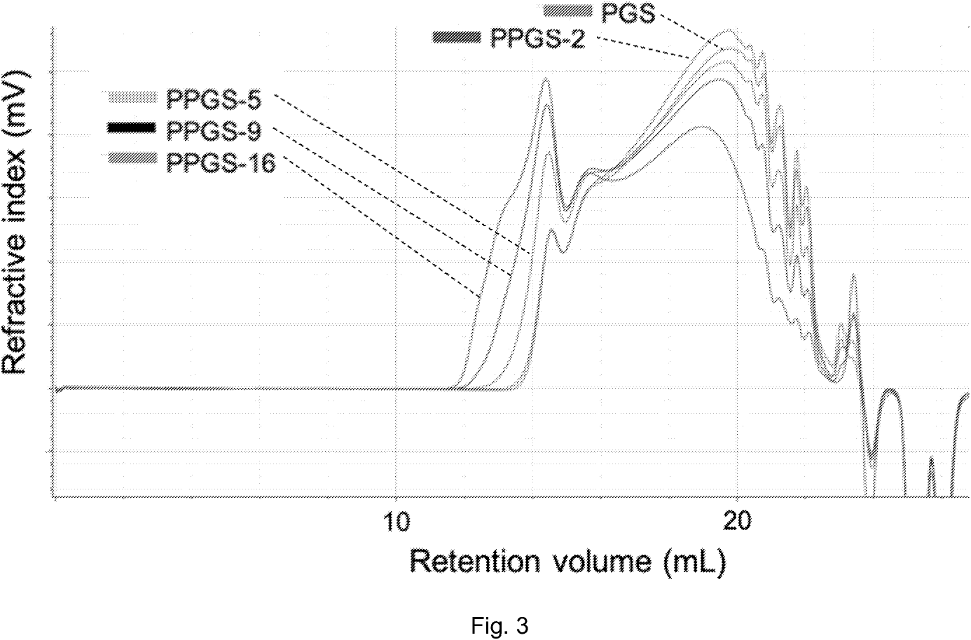

The molecular weights of these PPGS pre-polymers were determined by gel permeation chromatography (GPC) (FIG. 3 and Table 1).

TABLE 1

Mₙ, M_{w} and PDI of the PGS and PPGS pre-polymers by GPC analysis

| Sample | Mₙ, Da | M_{w}, Da | PDI |
|---|---|---|---|
| PGS | 4110 ± 163 | 24369 ± 493 | 5.95 ± 0.14 |
| PPGS-2 | 4293 ± 75 | 24661 ± 245 | 5.74 ± 0.06 |
| PPGS-5 | 4874 ± 42 | 44853 ±359 | 9.20 ± 0.05 |
| PPGS-9 | 5816 ± 82 | 111763 ± 1414 | 19.21 ± 0.04 |
| PPGS-16 | 7970 ± 7 | 303476 ±6481 | 38.08 ± 0.8 |

The GPC analyses demonstrated a steady increase in the number average molecular weight (Mₙ) from 4110 ± 163 Da for the PGS to 7970 ± 7 Da for the PPGS-16. However, the weight average molecular weight (M_{w}) and polydispersity (PDI) were significantly increased in PPGS-5 (44850 ± 360 Da, 9.2 ± 0.05), PPGS-9 (111760 ± 1414 Da, 19.2 ± 0.04) and PPGS-16 (303480 ± 6480 Da, 38.1 ± 0.8) compared to the starting PGS (24370 ± 490 Da, 5.95 ± 0.14). Some of the palmitic anhydrides in the reaction appeared to have driven the PGS pre-polymer to further condense and yield some high molecular weight chains. Taking the PPGS-16 as an example, according to the molecular weight of the repeat unit and the measured M_{w} of the starting PGS (24370 ± 490 Da), supposing that all of the free hydroxyl groups on the repeats were coupled with palmitate moieties (namely 100% functionalization), the theoretical M_{w} of the resultant PPGS would be approximately 47000 Da. In contrast, the tested M_{w} of the PPGS-16 with only 16 mol.% of palmitate pendants already reached 303480 ± 6480 Da with PDI of 38.1 ± 0.8. The GPC data suggest that a further condensation indeed occurred between the PGS chains, which yielded a fraction of high molecular weight pre-polymers and significantly increased the molecular mass distribution. It is likely that some of the palmitic anhydrides reacted with free carboxylic acid groups on the PGS pre-polymers to form unsymmetrical mixed anhydrides in the presence of the triethylamine catalyst in the reactions. These unsymmetrical anhydrides on the PGS were then esterified with free hydroxyls from adjacent PGS chains. As a result, some high molecular weight pre-polymers were generated by such condensation reactions. The fraction of high molecular weight PPGS pre-polymers increased accordingly with the palmitic anhydride contents in the reactions as the GPC spectra demonstrate (FIG. 3). As the proton NMR analyses demonstrated, only 40 - 50% of the palmitic anhydrides were reacted to become pendent on the PPGS-2 to PPGS-16 under the current reaction conditions (FIG. 1A). It is speculated that most, if not all, of the rest palmitic anhydrides were consumed in the condensation reactions.

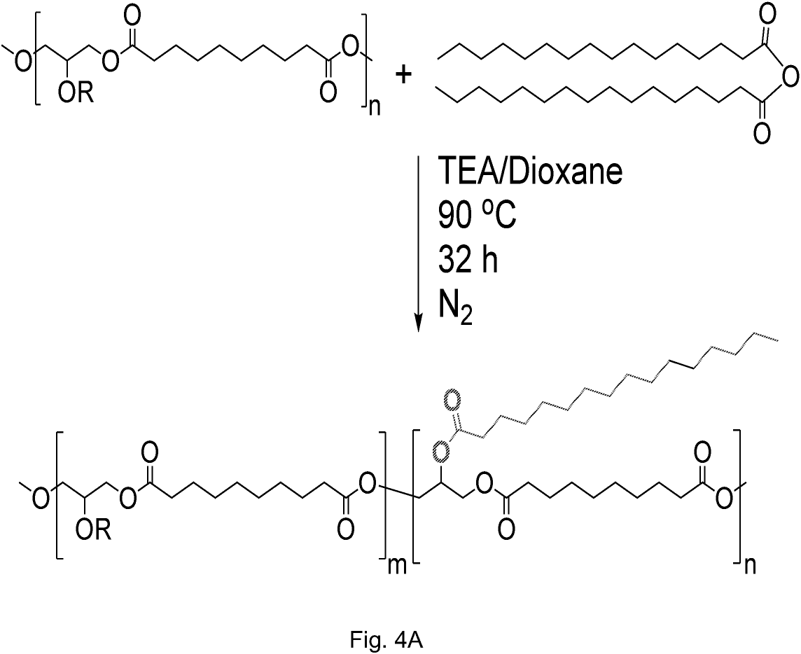

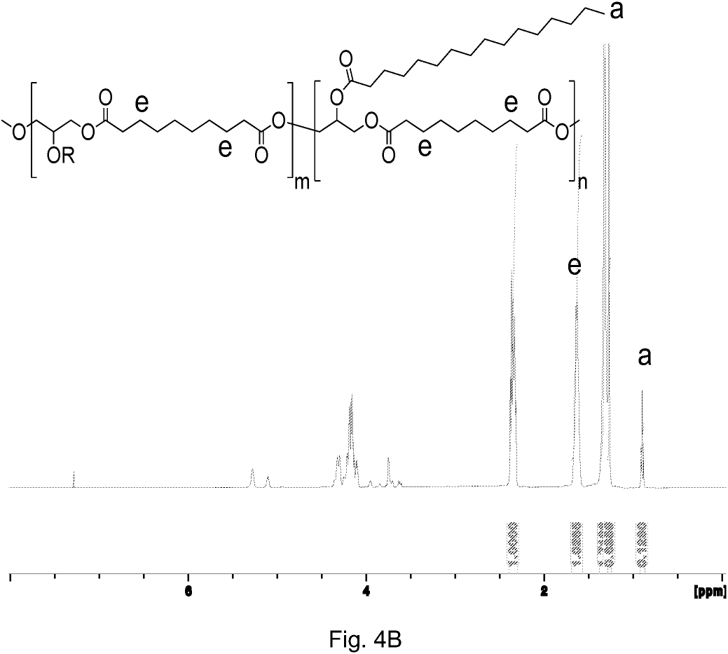

To examine this speculation, another experiment was performed by reacting the PGS with 40 mol.% of palmitic anhydrides (relative to the PGS repeats) for 32 h versus the initially 20 h (FIG. 1A, FIG. 4A). The actual palmitate pendants remained at 16 mol.% in the resultant PPGS (FIG. 4B), similar to the result of the 20 h reaction (FIG. 2D). This experiment indicated that increase of the reaction time could not immobilize more palmitates on the PPGS and thus confirmed the above speculation. Therefore, the high molecular weight PPGS pre-polymers were formed in proportion to the contents of the palmitic anhydrides in the reactions (FIG. 3).

In the following sections, these pre-polymers are thermally crosslinked and used to investigate the impacts of different palmitate contents on the materials properties.

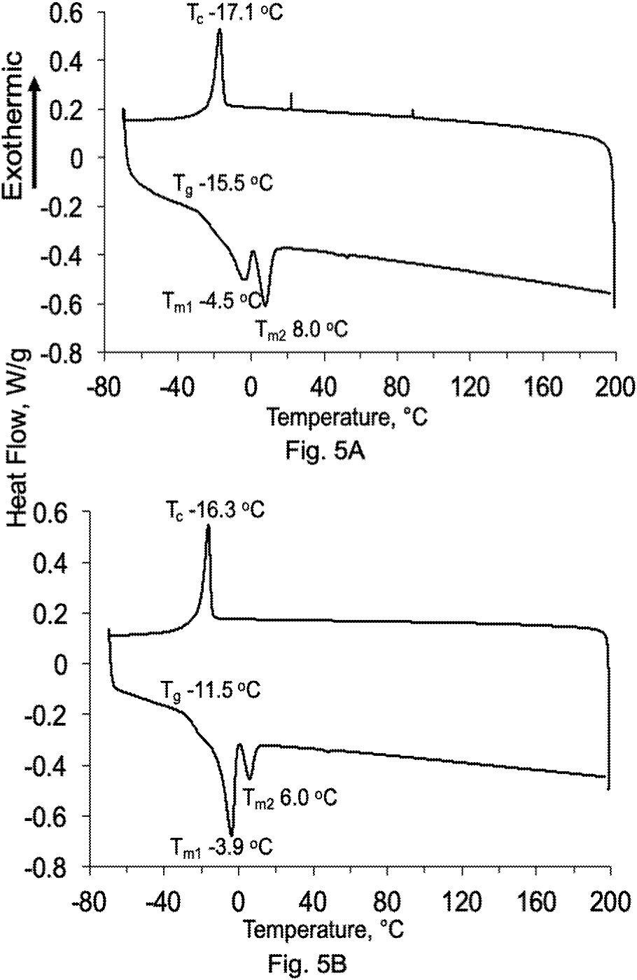

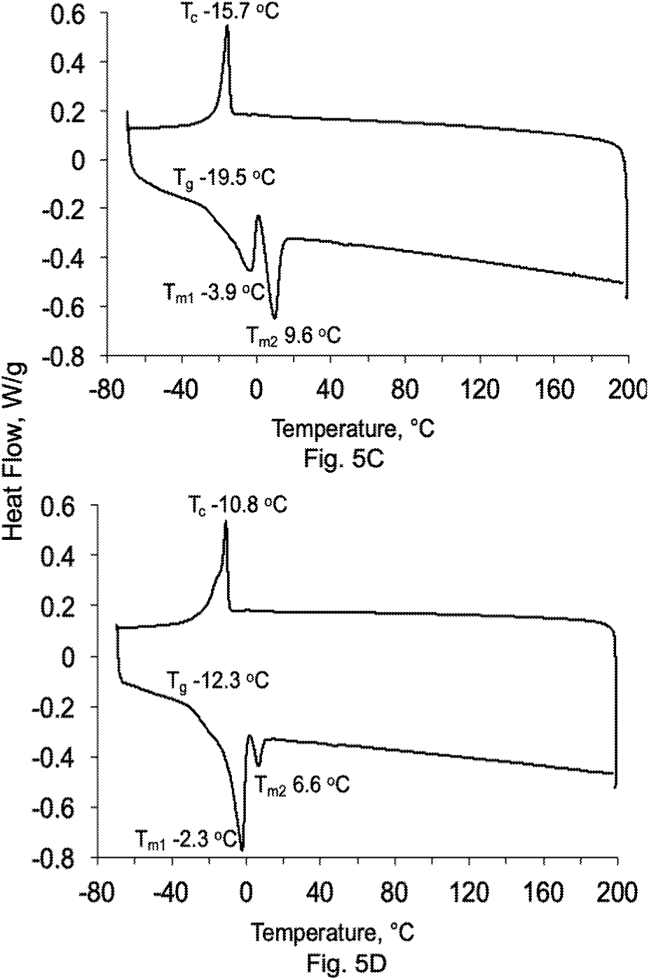

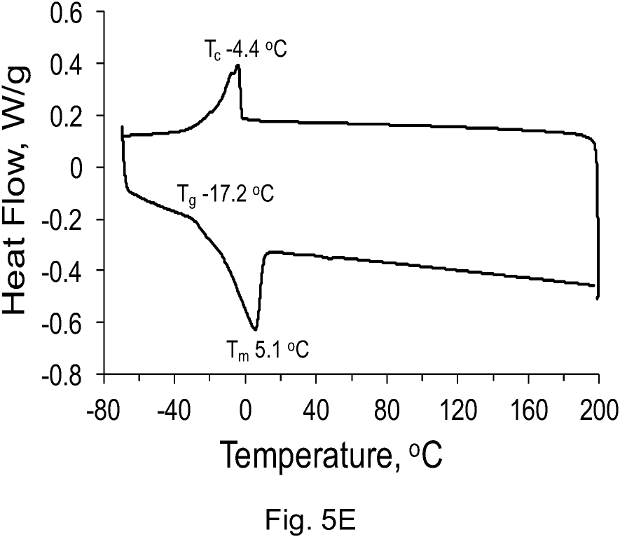

Palmitate pendants alter the crystallization patterns and thermal properties. DSC analysis was used to examine the effects of palmitate pendants on polymer chain packing, mobility, crystallization and thermal properties of the PPGS pre-polymers (FIGS. 5A-5E). All elastomers demonstrated three main thermal events of glass transition (T_{g}), melting (Tₘ) and crystallization (T_{c}) upon the heating and cooling processes. All of these thermal events occurred at temperatures below 10° C. The data indicated that the PPGS pre-polymers are semi-crystalline materials which reach a rubbery elastic state when the temperature is above 10° C. During the cooling process, the T_{c} and exothermic enthalpies increased correspondingly with the palmitate contents (FIGS. 5A-5E and Table 2).

TABLE 2

The crystallization, glass transition and melting temperatures and the associated enthalpies from the DSC analysis_{<}

| Sample | T_{c}, °C, -ΔH_{C} J/g | T_{g}, °C | Tₘ₁ °C, ΔHₘ₁ J/g | Tₘ₂ °C, ΔHₘ₂ J/g |
|---|---|---|---|---|
| PGS | -17.1, 18.5 | -15.5 | -4.5, 10.3 | 8.0, 8.2 |
| PPGS-2 | -16.3, 20.2 | -11.5 | -3.9, 19.6 | 5.9, 3.9 |
| PPGS-5 | -15.7, 21.0 | -19.5 | -3.9, 20.3 | 9.6, 16.4 |
| PPGS-9 | -10.8, 25.1 | -12.3 | -2.3, 26.4 | 6.5, 2.8 |
| PPGS-16 | -4.4,30.2 | -17.2 | 5.1,34.8 | / |

For example, the T_{c} elevated from -17.1° C. for the PGS to -4.4° C. for the PPGS-16, demonstrating an easier crystallization as the palmitate content increases. The exothermic enthalpies are considered as the energies released from the crystallization process. The measured values gradually increased from 18.5 J/g for the PGS to 30.2 J/g for the PPGS-16. Therefore, the palmitate pendants facilitated the crystallization and introduced additional crystalline structures in these PPGS elastomers compared to the PGS alone. In addition, the heat flow curves changed from a relatively sharp peak for the PGS to a broad peak with a toe, particularly for the PPGS-9 and PPGS-16. These data indicated that the palmitate pendants have altered the crystallization patterns. Next, the heating process and the associated endothermic events will be discussed to further explore the glass transition and the crystalline properties in these elastomers.

Corresponding to the crystallization is the melting process and the associated temperature and enthalpies. With the palmitate pendants below 9 mol.%, all of the PPGS elastomers and the PGS control showed two melting temperatures (Tₘi and Tₘ₂), reflecting two types of crystalline structures formed in these elastomers (FIGS. 5A-D and Table 2). When the palmitate pendants reach 16 mol.%, the Tₘ merged into one at 5.1° C. in the PPGS-16 (FIG. 5E). It is worth noting that the first melting temperature (Tₘi) and the associated enthalpies (AHₘi) elevated accordingly with the palmitate contents (Table 2). However, the impacts on the second melting temperature (Tₘ₂) and the associated enthalpies (AHₘ₂) show a fluctuation pattern from the PPGS-2 to PPGS-9 (Table 2). Interestingly, the sum of AHₘi and AHₘ₂ in the PGS control equaled to the released enthalpies during the crystallization process (-ΔH_{C}, 18.5 J/g). To all other PPGS elastomers, the endothermic enthalpies (sum of AHₘi and AHₘ₂) were slightly higher than the exothermic enthalpies (-AH_{C}), except the PPGS-5 with a relatively large difference (Table 2). The differences of the enthalpies across the heating and cooling processes were likely caused by the relaxation of polymer chain motion because of the interference of the palmitate pendants. Such interference is not present in the PGS control. The interference on the polymer chain motion and impacts on the amorphous structures formed in these elastomers are further elucidated by the variation of the glass transition.

The T_{g} reflects the glass transition of amorphous structures arrested in these elastomers. The T_{g} demonstrated repetitive increase and decrease with the palmitate pendants ranged from 0 to 16 mol.% (FIGS. 5A-5E and Table 2). Generally, the T_{g} reflects the chain flexibility in the polymer networks. In the present case, the PPGS-5 possessed a lowest T_{g} at -19.5° C., suggesting an easiest chain motion among these elastomers, followed by the PPGS-16 with T_{g} aₜ -17.2° C_{.} In contrast, the PPGS-2 and PPGS-9 with T_{g}s of -11.5° C. and -12.3° C. were both higher than the PGS control at -15.5° C., suggesting a hindered chain motion. The T_{g} variations were indicative to the ease of the polymer chain mobility and implied different microscopic structures formed in these elastomers. Combined with the difference between the melting enthalpies (ΔH_{mI} and AHₘ₂) and the crystallization enthalpies (-AH_{C}), the PPGS-5 and PPGS-16 with lower T_{g}s appeared to be more prone to form more crystalline structures than the PPGS-2 and PPGS-9 during the cooling process. As discussed, in the crystallization process, the palmitate pendants introduced additional crystalline domains and facilitated the crystallization, which tends to hinder the polymer chain motion. However, the palmitate pendants could also prevent the polymer chains from close packing that likely increase free spaces for the chain motion and organization. Therefore, the two opposite effects from the palmitate pendants compete for control of the microstructures and thermal properties of these elastomers in a content-dependent manner. These microstructures and thermal properties will in turn affect their mechanical properties and degradation profiles.

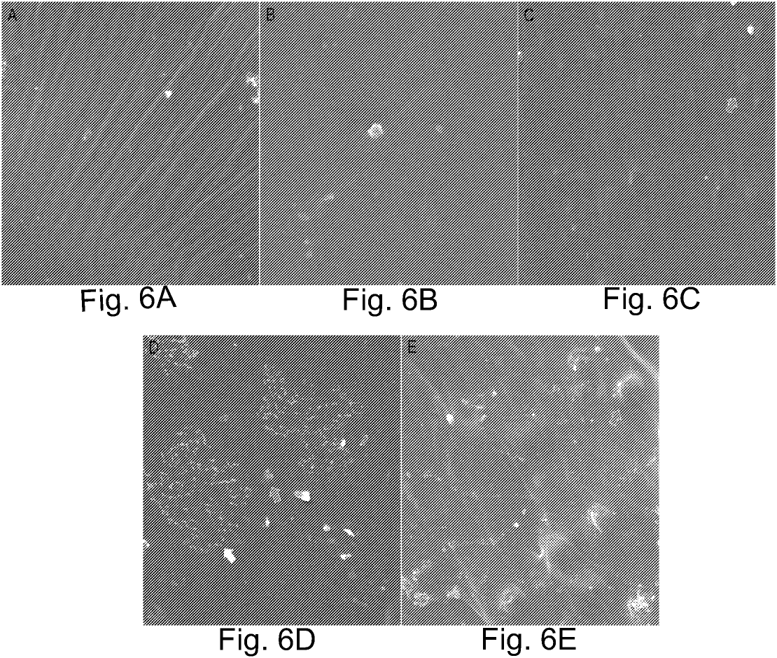

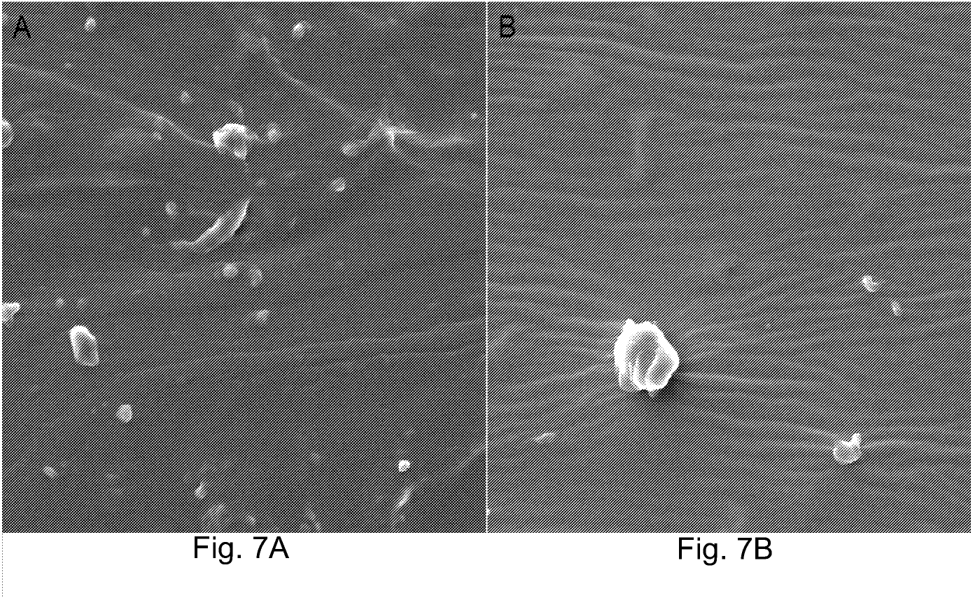

Palmitate contents affect the microstructures inside the elastomers. To further examine the microstructures formed in these elastomers, SEM was used to observe the microscopic morphologies at their cross-sectional areas (FIGS. 6A-6E). Interestingly, the PGS control showed waving structures at the cutting interface (FIG. 6A), while both the PPGS-5 and PPGS-16 elastomers demonstrated protruded micro-islands across the cross sections (FIGS. 6C, 6E). They appeared like microparticle-doped domains distributed throughout the cross sections. Compared to the PPGS-5, the PPGS-16 possessed micro-islands with a larger diameter. In contrast, the PPGS-2 and PPGS-9 both possessed relatively smooth interfaces across the cross sections (FIGS. 6B, 6D). Interestingly, the PPGS-9 showed another type of self-assembled micro-patterns at the interface with sparse micro-islands (FIGS. 6A, 6D). Little micro-island or self-assembled micro-pattern were observed in the PPGS-2 (FIG. 6B). These morphologies at the cross sections were not formed by the cutting process. The SEM micrographs indicated that different palmitate contents drove the polymer chains to assemble into different micro-domains dispersed in these elastomers. The 5 and 16 mol.% of palmitate pendants tended to make the polymer chains to form microparticle-doped domains throughout the networks, whereas the 9 mol.% palmitate pendants promoted the polymer chains to form micro-patterns in the PPGS-9. These micro-islands and micro-patterns were unlikely formed by free palmitates which potentially resided in these PPGS elastomers. To this end, two additional elastomers were prepared by physically mixing PGS pre-polymer with free palmitic acid at 9 mol.% and 16 mol.%, respectively. The mixtures were then crosslinked by the same crosslinking conditions to form two elastomers, PPGS-Mix-9 and PPGS-Mix-16. Neither micro-patterns nor micro-islands were observed in these two elastomers (FIGS. 7A-7B). These data suggest that immobilized palmitate pendants could drive the polymer chains to form different microstructures, but the physically mixed palmitates could not. Although few micro-pattern or microparticle were observed in the PPGS-2, the 2 mol.% palmitate pendants appeared to have contributed to smooth the polymer interfaces. In addition, the micro-patterns observed in the PPGS-9 do not cause additional roughness at the interface. It is interesting to note that the hydrophobic palmitate pendants drive the polymer chains to assemble into different microstructures depending on the contents, although the exact mechanism is unknown. The irregular morphological changes inside these elastomers are likely affected by multiple factors: palmitate content, molecular weight and the fraction of high molecular weight PPGS (FIG. 3). Overall, the PGS, PPGS-5 and PPGS-16 elastomers form rougher interfaces than the PPGS-2 and PPGS-9, which make the former three elastomers to possess more free spaces inside the networks. This is likely one of the reasons why the PGS, PPGS-5 and PPGS-16 have lower T_{g}s than the PPGS-2 and PPGS-9 as the DSC analysis demonstrated. The different microstructures and spaces inside these polymer networks would eventually affect their degradation profiles as they provide different pathways for water molecule penetration.

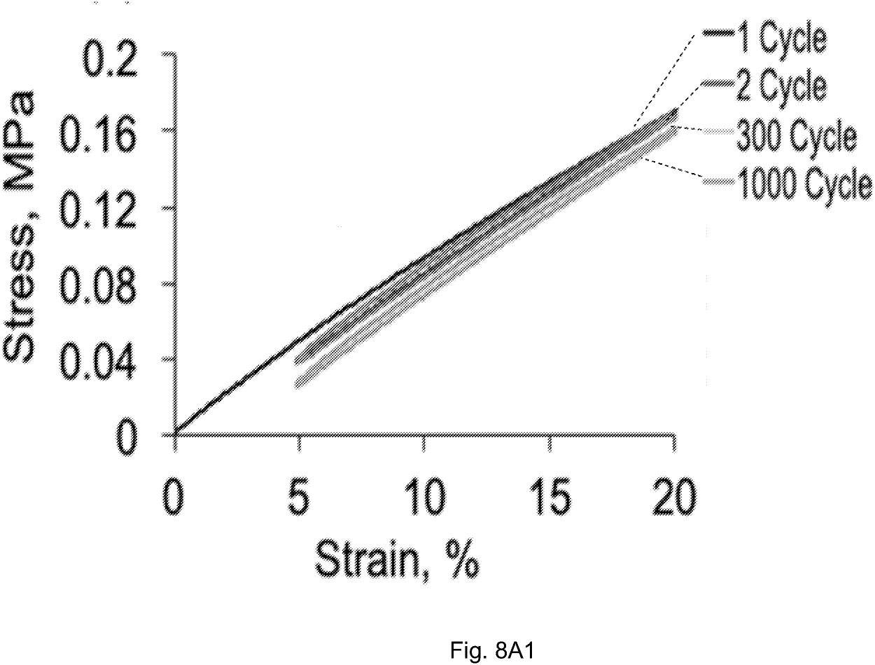

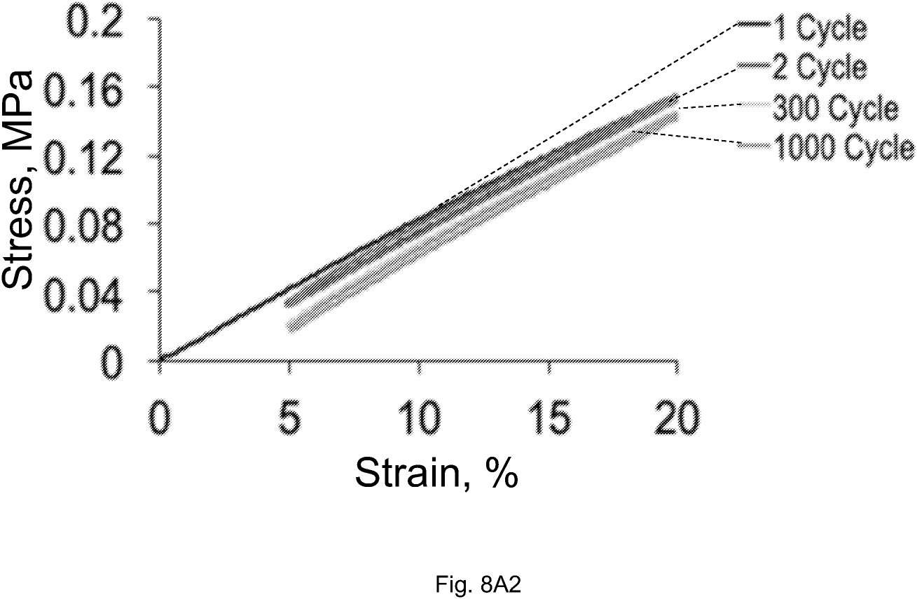

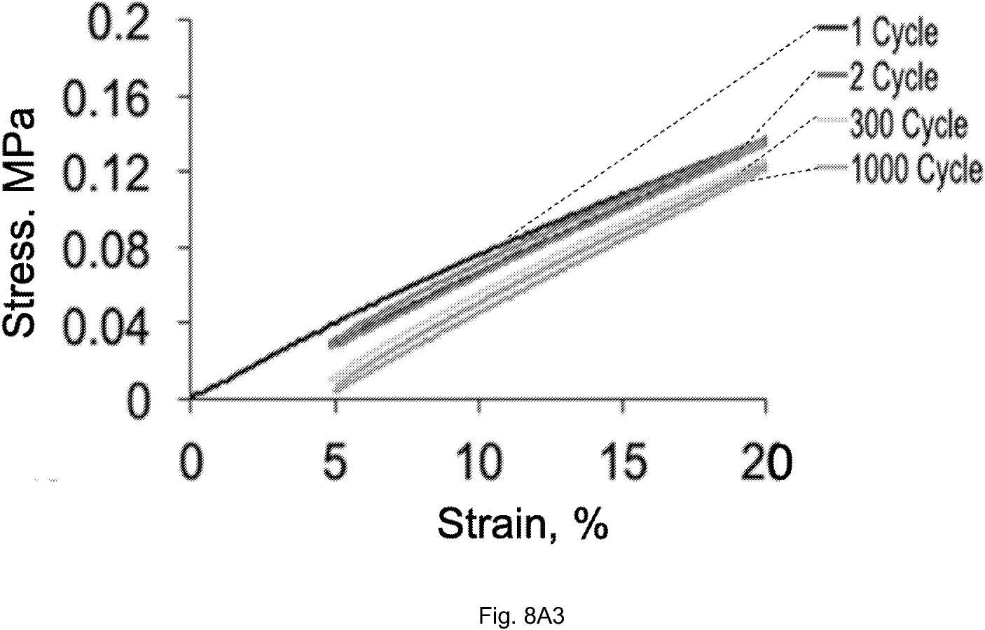

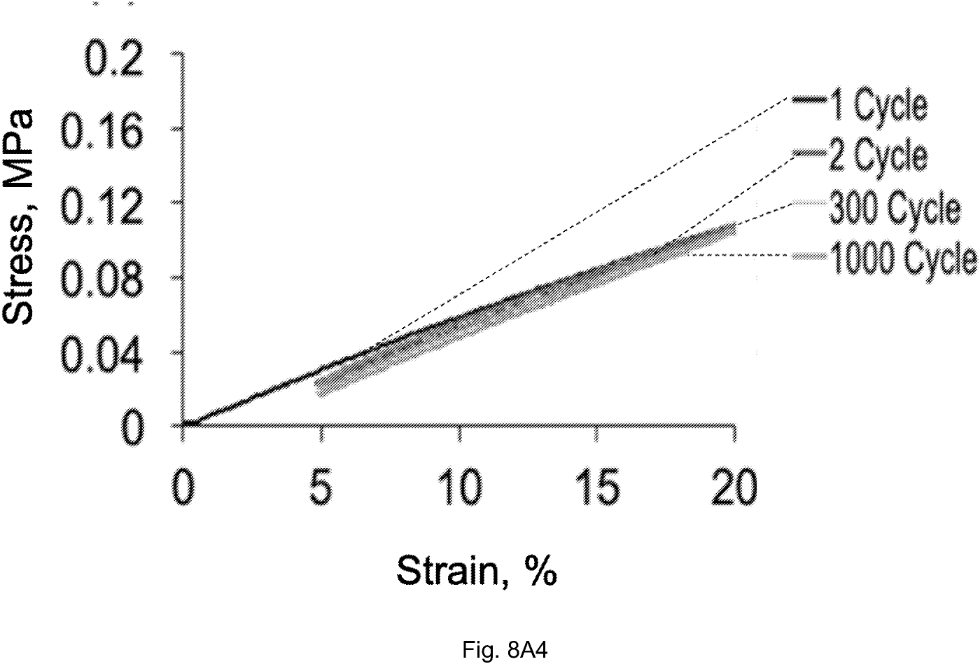

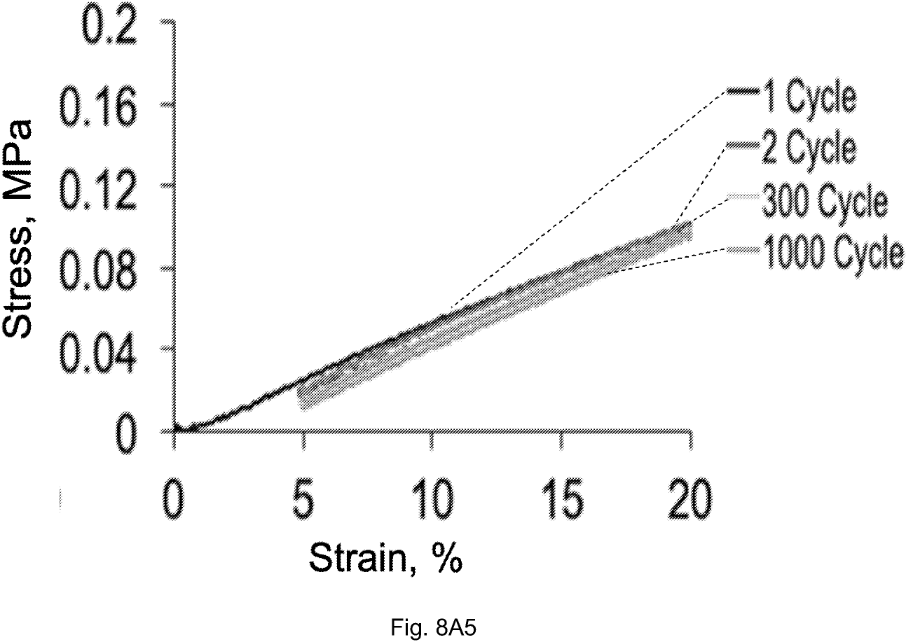

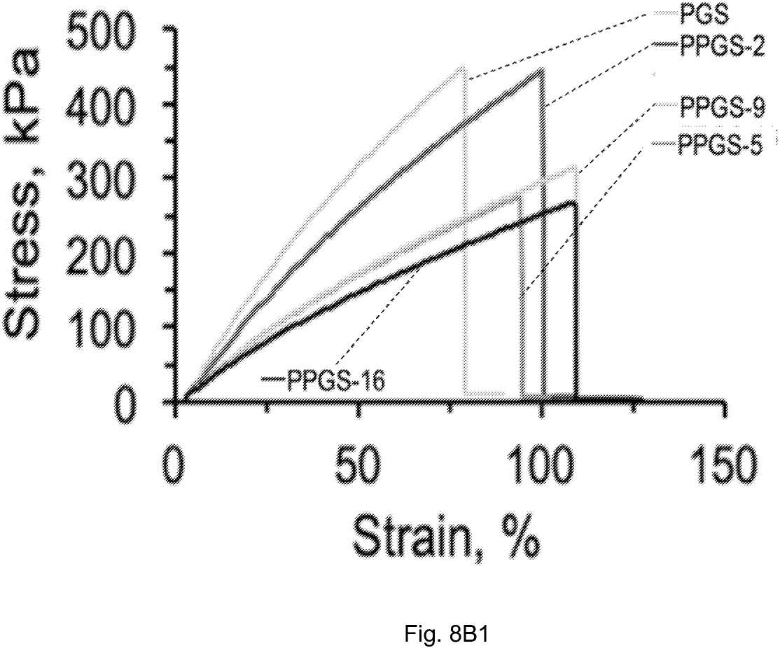

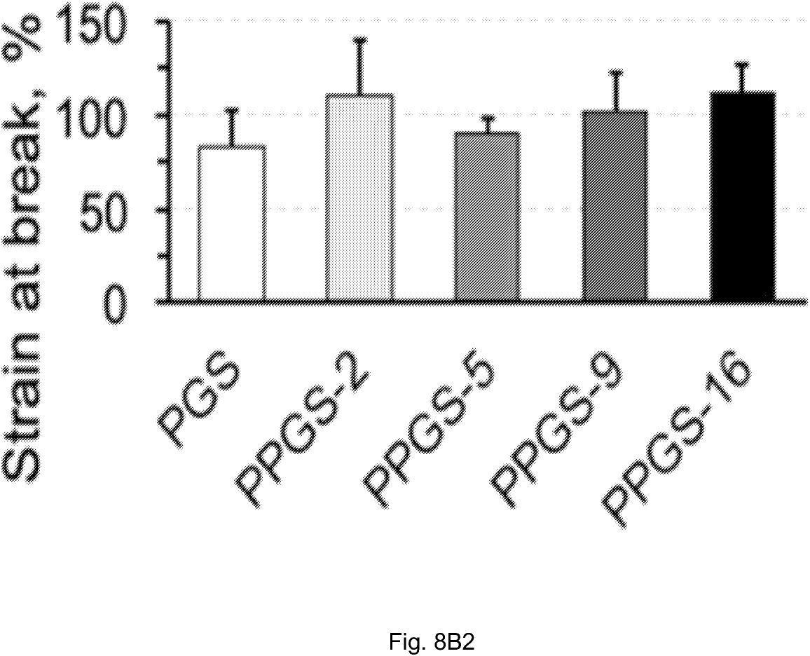

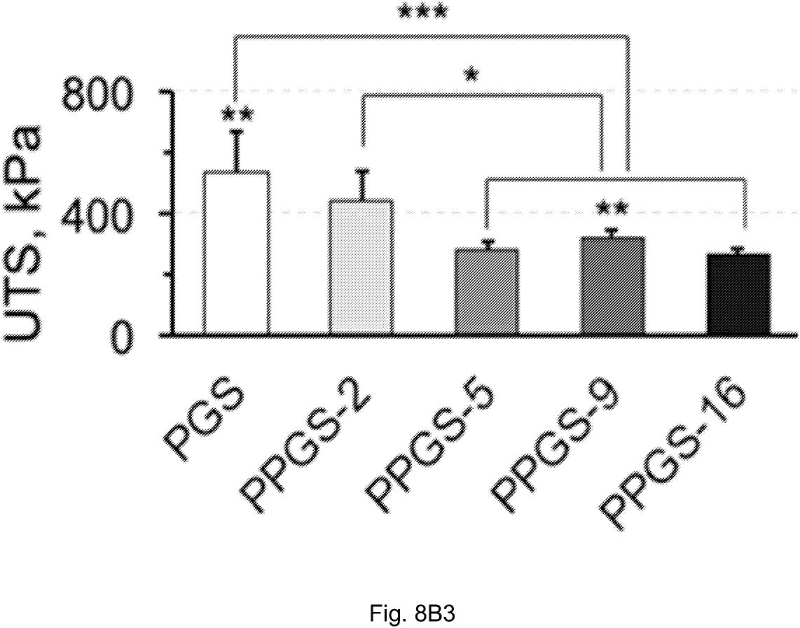

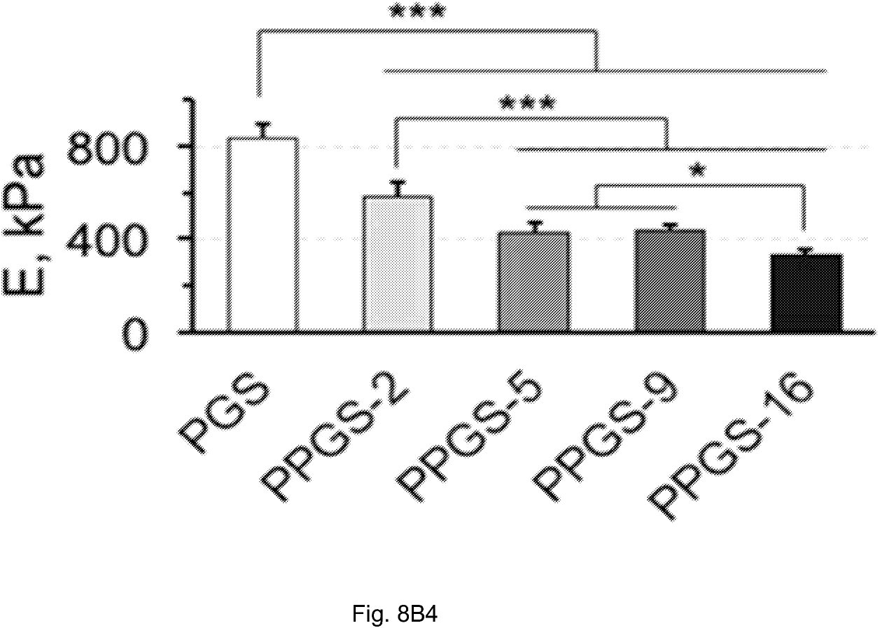

Palmitate pendants mediate the elasticity and mechanical properties. Elasticity was used to represent the ability of the elastomers to undergo reversible elastic deformations. The elasticity is an important property for an elastomeric scaffold to retain its integrity in a mechanically dynamic environment and provide sustainable mechanical support and physical cues for tissue regeneration. To examine the elasticity, cyclic tensile tests were performed on dog-bone samples of these elastomers (FIG. 8A1-8A5). The loading speed was set at 30 mm min⁻¹ with strain between 5% and 20%. The reversible deformations were tested at this strain range because many soft tissues such as arteries and ligaments typically undergo biomechanical deformations within this range. Notably, all elastomers could sustain the elastic deformations for at least 1000 cycles without failure. The PGS control, PPGS-2 and PPGS-5 elastomers demonstrate small hysteresis loops over the cyclic loading. The hysteresis loops appeared to have increased gradually from the PGS control to the PPGS-2 and PPGS-5, respectively. The elastic deformation profiles of these three elastomers indicate somewhat damages of their polymer networks by the cyclic loading. Interestingly, the PPGS-9 and PPGS-16 demonstrate less hysteresis loops across the cyclic tensile tests, indicating little damage in the two polymer networks. The cyclic tensile tests suggest that the palmitate pendants with 9 and 16 mol.% enhance the elasticity, whereas 5 mol.% or lower contents show somehow opposite effects on the elastic performance. Without the palmitate pendants, the elasticity of the PGS elastomer is dependent on coiled polymer chains between the crosslinks and hydrogen bonds between the free hydroxyls and carbonyl groups (FIG. 1C1). The exerted stress from the cyclic loading is dissipated by the reversible movements of the polymer chains and the hydrogen bonds. Different with the PGS, the PPGS elastomers bear palmitate pendants which impart additional weak interactions such as hydrophobic interactions and crystalline domains (FIGS. 5A-5E, 6A-6E), although the hydrogen bonds are partially compromised by the palmitate pendants (FIG. 1C2). On the other hand, the different amounts of the palmitate pendants form the different crystalline structures and microstructures inside the polymer networks (FIGS. 5A-5E, 6A-6E, Table 2). These features synergistically play roles in mediating the elasticity and dissipating the stress from the reversible deformations. Collectively, the PPGS-9 and PPGS-16 showed enhanced elasticity, whereas the PPGS-2 and PPGS-5 slightly compromise the elastic performance. To compare other mechanical properties, uniaxial tensile tests were performed to further examine the strain at break, ultimate tensile strength (UTS) and Young’s modulus (E) related to the palmitate contents (FIG. 8B1-8B4).

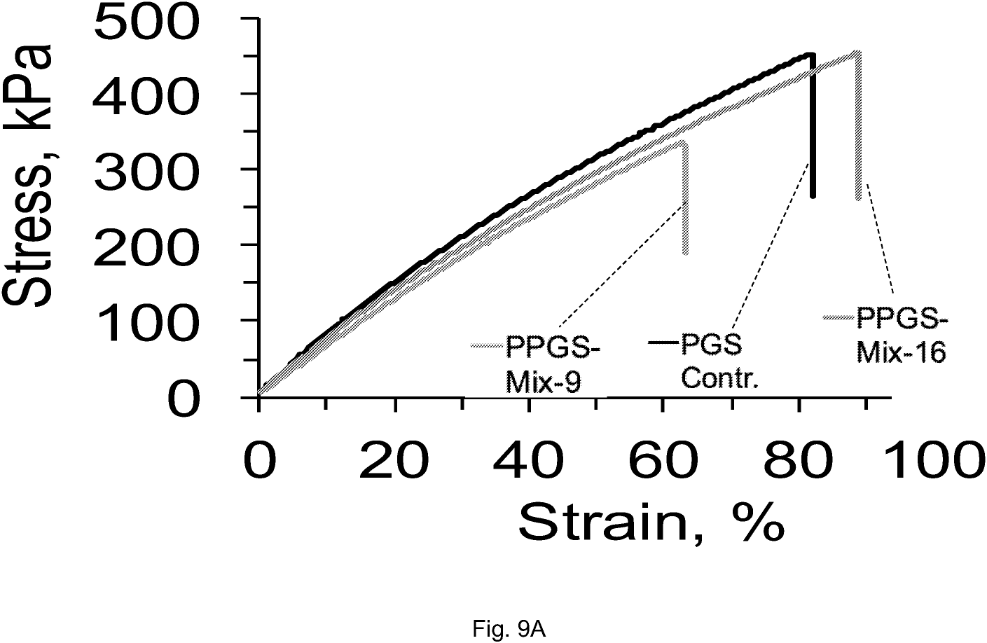

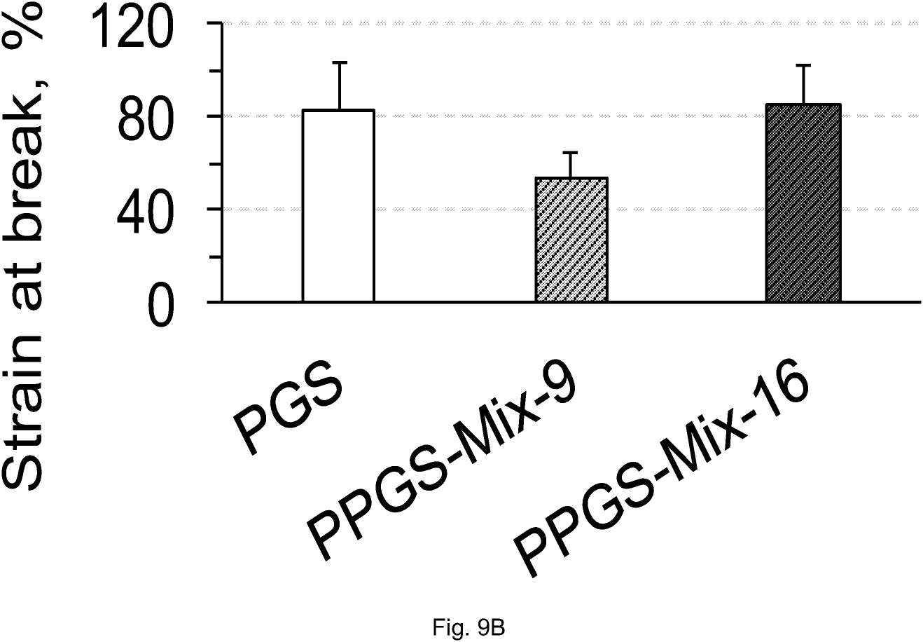

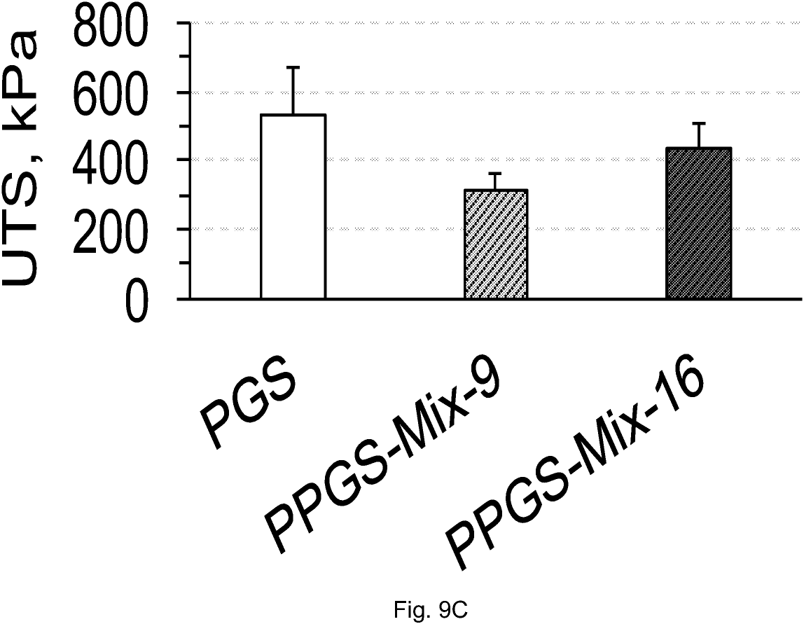

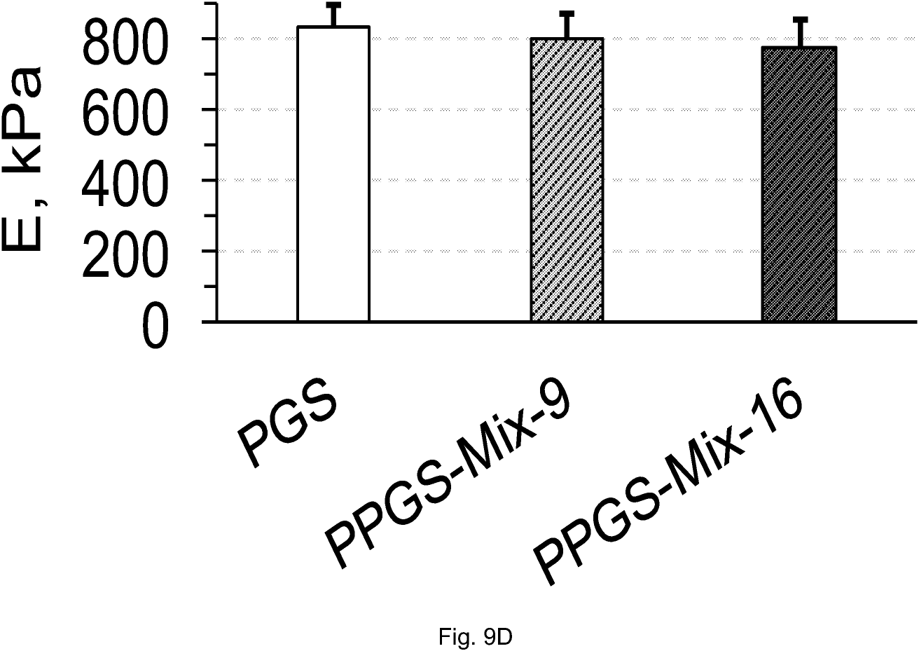

The uniaxial tensile tests indicate that the strain at break show a slight increase from the PGS to PPGS-16, but not statistically different (FIG. 8B2). The strain at break depends on the crosslinking densities and the polymer chain length between the crosslinks. Although the hydroxyl groups on the PPGS pre-polymers have been partially immobilized with the palmitate pendants (maximal 16 mol.%), the majority of the hydroxyls are free for crosslinking to form ester bonds. Therefore, under the same crosslinking conditions (150° C. for 24 h in a vacuum oven), the crosslinking densities are assumed to be similar among these elastomers. Given a gradual increase of the Mₙ from the PGS to PPGS-16 pre-polymers (FIG. 3 and Table 1), the polymer chain length between the crosslinks is therefore increased accordingly from the PGS to PPGS-16 elastomers. The change of the strain at break appeared in such a trend in these elastomers. Notably, the E values demonstrated a tunable fashion along with the palmitate contents (FIG. 8B4). The more palmitate pendants led to a softer elastomer. For example, the E values varied from initially 838 ± 55 kPa for the PGS to 582 ± 64 kPa for the PPGS-2, 426 ± 47 kPa for PPGS-5 and 441 ± 26 kPa for PPGS-9, and further reduced to 333 ± 21 kPa for the PPGS-16. Because these elastomers underwent a linear elongation, the UTS values were dependent on the strain at break and the E value. The UTS values accordingly reduced from 533 ± 137 kPa for the PGS control to 265 ± 18 kPa for the PPGS-16, similarly tunable along with the palmitate contents (FIG. 8B3). It is well established that the E of a thermoset elastomer is typically dependent on both the chemical and physical crosslinks formed in the polymer networks. In the present case, the chemical crosslinks were similar among them because of the same crosslinking conditions. The E values were therefore mainly tuned by the palmitate pendants. As discussed above, the palmitate pendants affected the polymer chain packing, mobility, crystalline structures and microstructures in a content-dependent fashion. These factors together affected the mechanical properties. For comparison, physically mixing palmitic acid with the PGS could not tune the mechanical properties of the elastomers (PPGS-Mix-9 and PPGS-Mix-16) like the palmitate pendants did (FIGS. 9A-9D). Overall, the increase of the palmitate pendants accordingly reduced the E and UTS and made the elastomer more compliant for the elastic deformations. Although the UTS was compromised, it would not affect the material applications in soft tissue engineering including the small arteries in this case.

The mechanical properties of the PGS alone and other PGS derivatives can also be tuned by changing the curing time and crosslinking densities. For example, a shorter crosslinking time typically forms less crosslinks and leads to a lower modulus and a longer elongation as we reported previously. However, less crosslinks would typically result in a quicker degradation of the elastomer, which is not desired for applications in small arteries. Preferably, the PPGS elastomer is softer with enhanced elasticity but slower in degradation than the PGS control to meet requirements for applications in small arteries. The mechanical data have demonstrated a partial achievement of these goals. Another objective is to slower down the degradation by the palmitate functionalities as is discussed in the next section.

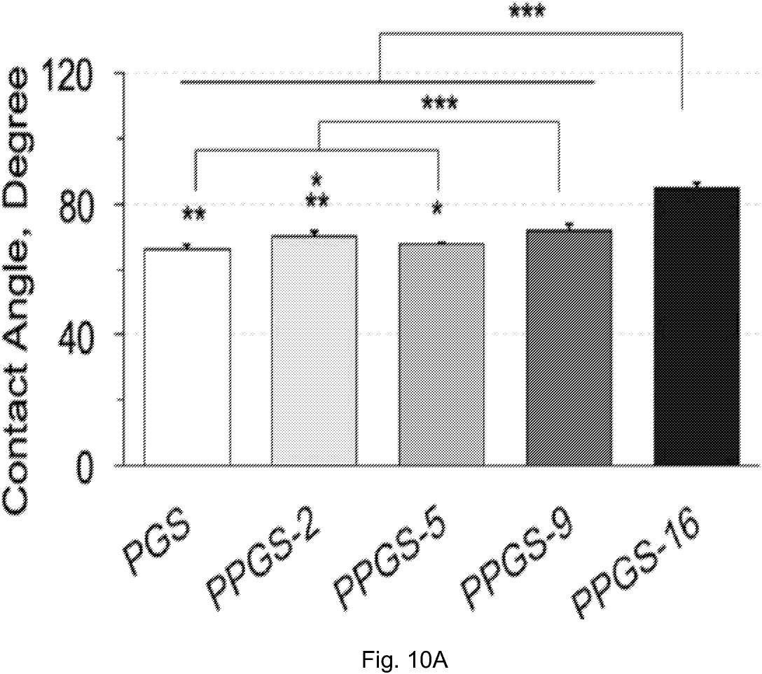

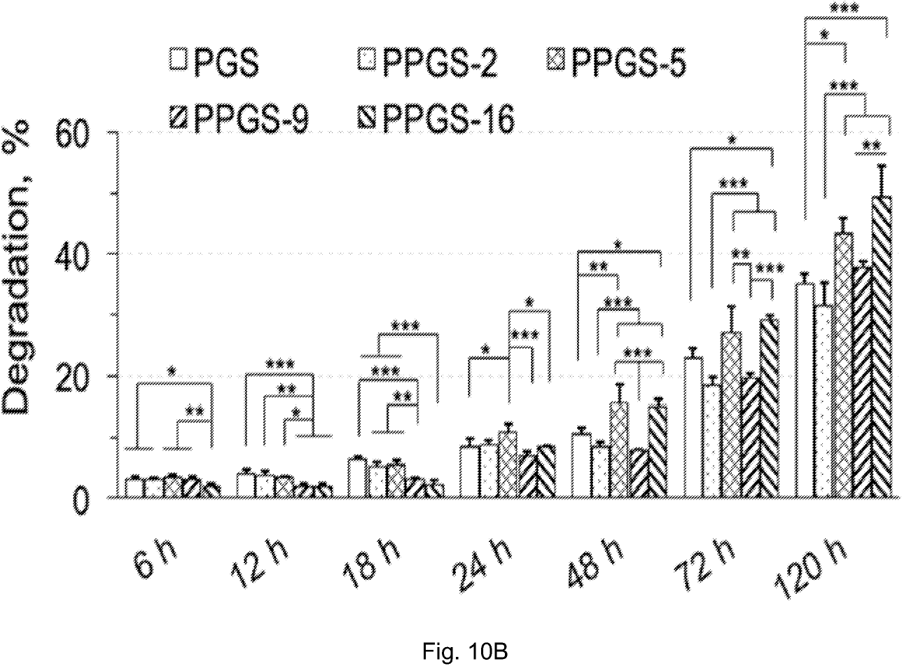

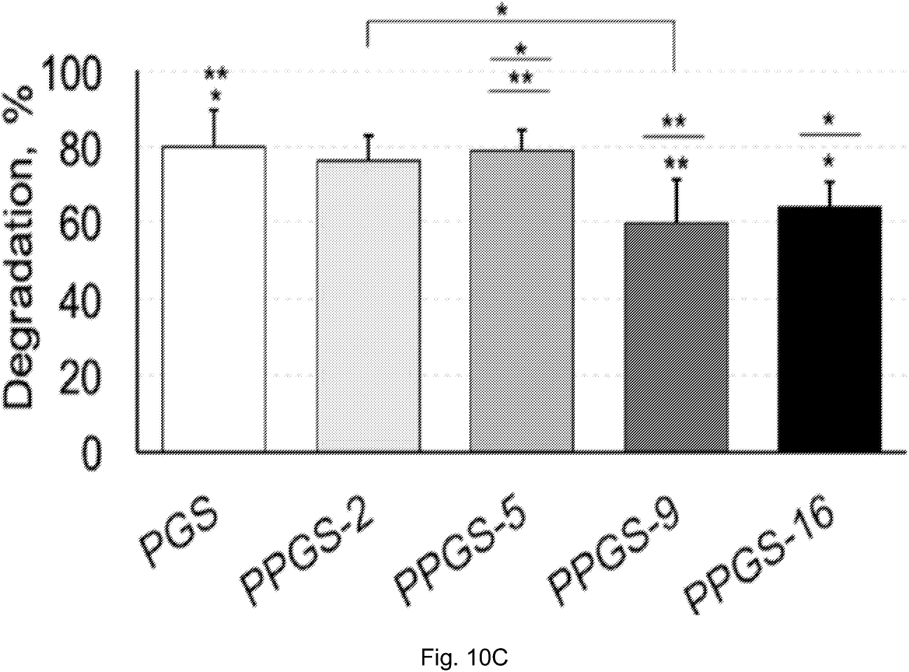

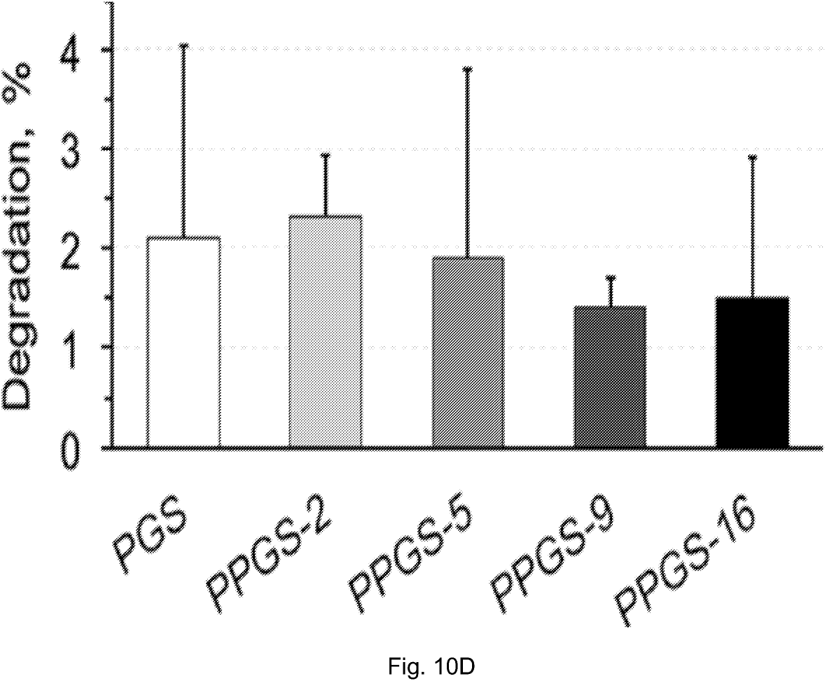

Palmitate functionalities affect the hydrophobicity and degradation profiles. The palmitate pendants bear hydrophobic alkyls that would inhibit water molecules to access the polymer networks and would thus be expected to slow down the degradation. To examine the change of hydrophobicity, water contact angle measurement was performed to evaluate the hydrophobicity relating to the palmitate contents (FIG. 10A). The water contact angles showed a significant increase in the PPGS-16 (84.8 ± 1.6°) and then the PPGS-9 (72.0 ± 1.8°), and a slight increase in the PPGS-2 (70.3 ± 1.5°) compared to the PGS control (66.3 ± 1.2°).

Surprisingly, no significant difference was observed between the PPGS-5 (67.5 ± 0.6°) and the PGS control, although the PPGS-5 has an actual palmitate content 2.5 times to that of the PPGS-2 (FIG. 1B1-1B2). The reason has yet to be exactly known. Nevertheless, the water contact angle measurements reflected an overall increase of the hydrophobicity with the palmitate contents in the PPGS elastomers. On the other hand, although the PPGS-16 is most hydrophobic among them, it still possessed a water contact angle of 84.8 ± 1.6°. This contact angle value reflected a considerable hydrophilicity of this elastomer, which is still favored for cell attachment and proliferation on the material surface. Next, the degradation profiles were examined of these elastomers affected by the palmitate pendants (FIGS. 10B-10D).

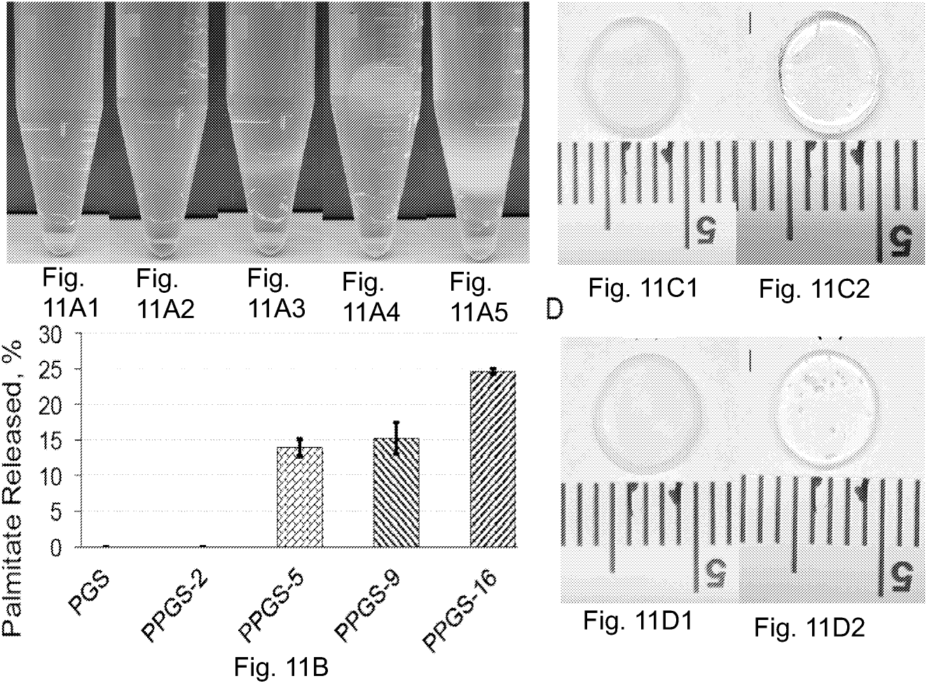

First an accelerated degradation test was performed in a 60 mM NaOH solution at 37° C. to quickly examine the effects of the palmitate contents on the degradation profiles (FIG. 10B). The PGS control showed a gradual increase of the degradation percent over the incubation time in the basic solution, following a typical surface erosion process similar to prior reports. Compared to the PGS control, the PPGS elastomers showed two degradation phases (FIGS. 11C1-11C2, and 11D1-11D2). For incubation times of less than 24 h, the degradation was inhibited by the hydrophobic palmitate functionalities and demonstrates the more hydrophobic PPGS elastomer with a slower degradation. For example, the PPGS-16 demonstrated the most significant inhibition on its degradation, followed by the PPGS-9 (FIG. 10B). The other two PPGS elastomers showed a slight, but not significant, inhibition on their degradations compared to the PGS control. At this stage, the surface erosion process dominated the degradation; the degradation profiles were consistent with the water contact angle measurements. However, for incubation times greater than 24 h, the degradation profiles changed. The PPGS-2 and PPGS-9 still slightly inhibited their degradations up to approximately 120 h incubation, but the PPGS-5 and PPGS-16 significantly facilitated their degradations between 48 and 120 h incubation. Therefore, the degradation went to the second stage as the incubation time exceeded 24 h (FIGS. 11C1- 11C2, and 11D1-11D2). At this stage, the water molecules and hydroxyl ions eventually entered into the polymer networks. The catalyzed hydrolysis occured to the ester bonds both on the polymer surface and inside the networks. The glycerol palmitates were cleaved and released from the PPGS samples (FIG. 11A1-11A5). The degradation pattern thus converted to a bulk erosion (FIGS. 11C1-11C2, and 11D1-11D2). This was also evidenced by the released palmitate-related components between 48 and 120 h incubation (FIGS. 11A1-11A5, and 11B). These palmitate-related compounds are amphiphilic molecules. They self-assembled into a separate layer of cloudy gel suspending in the basic solution above the PPGS-5, PPGS-9 and PPGS-16 samples (FIG. 11A1-11A5). No such gel was observed in the PGS control and PPGS-2 samples because they contained no or low content of the palmitate pendants.

In principle, all PPGS elastomers should have facilitated the degradation rate at the later stage since they all converted to the bulk erosion due to the cleavage of the palmitate pendants inside the networks. However, only the PPGS-5 and PPGS-16 showed acceleration while the PPGS-2 and PPGS-9 still demonstrated certain inhibitions on the degradations compared to the PGS control until 120 h (FIG. 10B). With respect to the DSC and SEM data, the PPGS-5 and PPGS-16 possessed lower T_{g}s with micro-islands at their interfaces, whereas the PPGS-2 and PPGS-9 had higher T_{g}s with relatively smooth interfaces compared to the PGS control (FIGS. 5A-5E, 6A-6E). The different rough interfaces suggested that water molecules and hydroxyl ions could access PPGS-5 and PPGS-16 polymer networks more easily. Therefore, the hydrolysis of the ester bonds and release of the palmitate components occured earlier in the PPGS-5 and PPGS-16 than the PPGS-2 and PPGS-9. This is likely why the PPGS-5 and PPGS-16 demonstrated a quicker degradation than other elastomers at the second stage. In addition, the PPGS-16 had more palmitate pendants to be cleaved and released than the PPGS-5, it therefore demonstrated the most degradation among them (FIGS. 10B, 11B). Notably, the rough interfaces affected the degradation behaviors of these elastomers at the second stage. The reason for the irregular variation in the interface roughness and T_{g} relating to the palmitate content is likely complicated by the factors of different palmitate content, molecular weight, and the fraction of high molecular weight PPGS (FIG. 3). The interplay of these factors affected the crystallinity, assembled micro-structures and interface roughness and consequently the degradation behaviors of these elastomers.

These materials will be used to make porous elastomeric scaffolds for utilities. Therefore, the degradation of the porous scaffolds with pore size of approximately 75 to 150 µm was evaluated in the 60 mM NaOH solution and PBS 1× (pH 7.4) at 37° C. respectively (FIGS. 10C-10D). This pore size was made to ensure the degradation solution easily entered into the scaffolds to keep the testing conditions consistent. Because the porous structures had a large surface area, the surface erosion dominated the degradation. In the basic solution, 76.3 ± 6.3% and 78.9 ± 5.3% of the PPGS-2 and PPGS-5 were degraded in 18 h, which was slightly less than the PGS control of 80.1 ± 9.5% but not significantly different. In contrast, the PPGS-9 and PPGS-16 degradations were reduced to 59.4 ± 11.2% and 63.7 ± 6.4% respectively, which were significantly less than the PGS control, but not significantly different between themselves. Their degradation rates were approximately 1.3 times slower than the PGS control. Here, the more hydrophobic PPGS-16 showed a slightly quicker degradation than the PPGS-9 likely because of the large surface area and more palmitate pendants cleaved from the PPGS-16. In contrast, these porous scaffolds were relatively stable in the PBS 1× (pH 7.4) at 37° C. All of these scaffolds demonstrated less than 2.3 ± 0.6% degradation over one month incubation. The degradation inhibitions by the palmitate pendants were similar to the trend observed in the basic solution, but no significant difference was observed because of very low degradation in the PBS.

Degradations of these elastomers with and without porous structures have been demonstrated in the basic and PBS solutions. The degradation tested in these conditions was mainly aimed to understand the impacts of the palmitate functionalities on the degradation kinetics of these elastomers. The in vivo environment will be significantly different from these tested conditions. The biodegradation will typically involve penetration of water molecules, absorption of protease, immune responses to the materials, and inflammatory cell infiltration, proliferation and secretion of catalytic molecules as well as the reactive oxygen species, among others. Regardless of these differences, it is anticipated that the in vitro degradation data could provide guidance to select the designed PPGS with suitable composition for different applications.

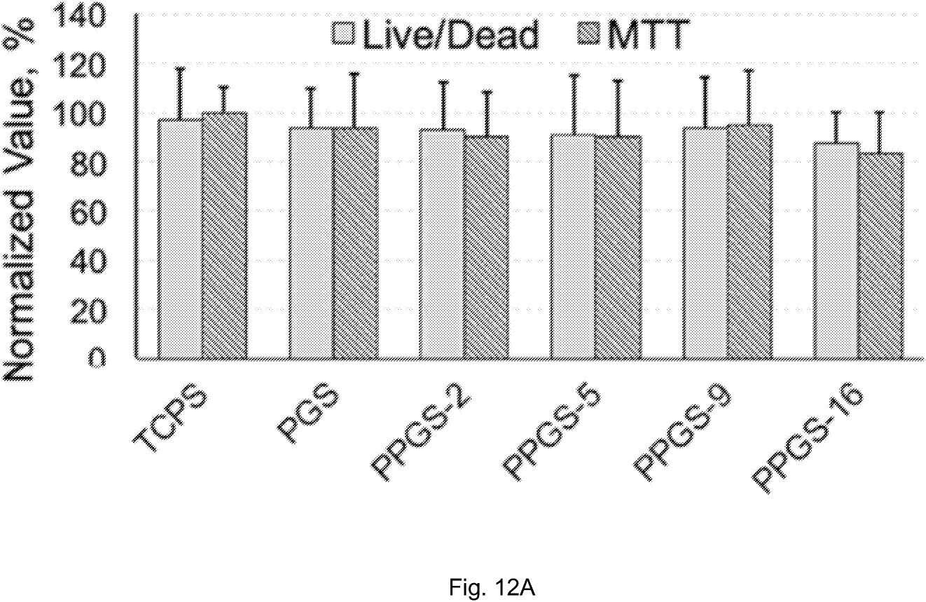

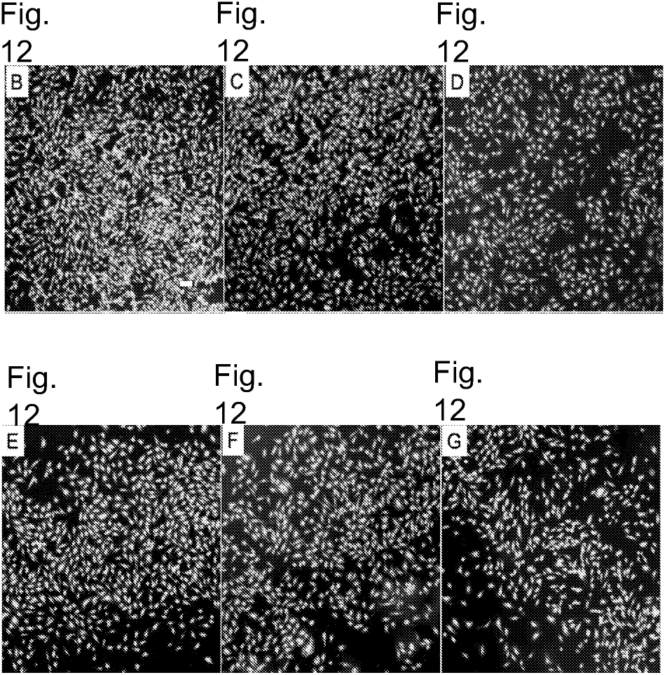

In vitro cytocompatibility. The cytocompatibility was evaluated by culturing HUVEC cells on the elastomers over 48 h. Live/dead and MTT assays were used to examine the cell viability and metabolic activities (FIGS. 12A-12G). Compared to the TCPS and PGS controls, the cellular metabolism and viability demonstrated only a slight but non-significant decrease on the PPGS-2 to PPGS-16 elastomers (FIG. 12A). The result indicated good cytocompatibility of all of these materials. The slightly compromised cellular metabolism and viability were likely induced by the increased hydrophobicity of these materials, but not toxicity. This was evidenced by the live/dead fluorescent micrographs (FIG. 12B). The cells could attach, spread and proliferate on these materials with similar morphologies, but the cell densities decreased slightly from the PGS to PPGS-16 with sparse dead cells (red stain). The reduction of the cell density appeared consistent with the increase of the hydrophobicity among these elastomers (FIG. 10A). Therefore, the increased hydrophobicity inhibited the cell proliferation on these elastomers and the cellular activities were accordingly compromised. Overall, the PPGS elastomers with palmitate contents up to 16 mol.% maintained good cytocompatibility of PGS.

Notably, the palmitate pendants up to 16 mol.% could adjust the modulus from 838 ± 55 kPa for the PGS to 333 ± 21 kPa for the PPGS-16. Such adjustment made the elastomers more compliant for elastic deformations with enhanced elasticity compared to the PGS control. The palmitate pendants up to this level also significantly increased the hydrophobicity and reasonably inhibited the degradation of the porous scaffold by nearly 1.3 times. On the other hand, the HUVEC cell growth on the PPGS remained nearly the same as on the PGS control. Because these PPGS elastomers were designed for constructing synthetic grafts for small arteries, the palmitate contents ranged from 2 to 16 mol.% were investigated. A higher palmitate content would likely induce a strong inflammatory response to the materials which is adverse for the arterial remodeling. According to the synergistic effects of the reduced elastic modulus, enhanced elasticity and slower degradation, it was deduced that PPGS-9 and PPGS-16 could meet the requirements for small arteries. PPGS-9 and PPGS-16 together with PGS where therefore used to construct vascular grafts for an in vivo study in a rat carotid model. In addition, these materials and the strategy reported here are also suitable for other soft tissue engineering applications. In other cases, the palmitate contents might need further adjustment to meet the specific applications.

The palmitate functionalization simultaneously adjusted the material hydrophobicity, crystallinity, thermal properties, degradation profiles and mechanical properties. The PGS derivatives with 9 and 16 mol.% palmitate pendants are softer elastomers with enhanced elasticity and reduced degradation rate. Palmitate is the most abundant saturated fatty acid in the body and the modified PGS retained good biocompatibility. It is expected that these derivatives will broaden the applications of PGS in soft tissue engineering.

Experimental methods. Synthesis of palmitate functionalized poly(glycerol sebacate) (PPGS) pre-polymers. All chemicals were used as received. Poly(glycerol sebacate) (Regenerez®, Secant Group LLC, PA) was reacted with different amounts of palmitic anhydride (TCI, >96.0%) to yield the resultant PPGS pre-polymers with different palmitate contents. Specifically, 50 mmol of PGS (12.92 g) (based on the repeat units) was dissolved in 50 ml 1,4-dioxane anhydrous (Spectrum, 99.8%) in a round bottom flask. Then 2.5, 5, 10 and 20 mmol of palmitic anhydrides was separately added to each batch of the PGS solutions, followed by adding additional 7.5, 15, 30 and 50 ml of dioxane anhydrous respectively. The mixture was dissolved at 50° C. for approximately 15 min, followed by adding triethylamine to each reaction at a molar ratio of 2:1 relative to the palmitic anhydride. The reaction was heated at 90° C. for 20 h with magnetically stirring under a nitrogen atmosphere. After reaction, the solution was cooled down to room temperature. Each product solution was precipitated in 900 ml of hexanes (Pharmco, ACS grade) with gently magnetic stirring overnight. The supernatant was decanted and the viscous residue was dissolved in 30 ml of acetone (Pharmco, ACS grade), followed by adding approximately 900 ml of hexanes again for precipitation with magnetically stirring overnight. Such solvation and precipitation were repeated twice, followed by washing with hexanes once. These pre-polymers were dried in a vacuum oven at 60° C. under reduced pressure for about 24 h to obtain the purified PPGS pre-polymers. In these four batches of reactions, the PPGS pre-polymers were prepared with theoretical palmitate contents of 5, 10, 20 and 40 mol.% relative to the PGS repeat units. The yields of them ranged from 96 %, 90 %, 86 % to 74 % as the palmitate contents increased. The actual palmitate contents were determined by proton NMR analysis to be 2, 5, 9 and 16 mol.%. These pre-polymers were assigned as PPGS-2, PPGS-5, PPGS-9 and PPGS-16. The immobilization efficiencies of the palmitic anhydrides were 40, 50, 45 and 40%, respectively.

Material characterizations. Proton NMR analysis (500 MHz, Bruker) was performed to record the chemical shifts of these PPGS pre-polymers. Samples were prepared by dissolving approximately 18 mg of each PPGS pre-polymer in 0.75 ml of CDC1₃ (Sigma-Aldrich). The integral area ratio of the protons at 0.895 ppm and 1.640 ppm was used to quantify the actual palmitate contents in these PPGS pre-polymers to be 2, 5, 9 and 16 mol.% (FIGS. 2A-2D).

The molecular weights were examined by gel permeation chromatography (GPC) using Malvern Panalytical OMNISEC GPC system (Malvern Instruments Ltd, UK). The OMNISEC advanced detection system is equipped with triple detectors, including refractive index, right angle and low angle light scattering (RI, RALS and LALS). Column set of T6000M and T3000 and tetrahydrofuran (THF, HPLC grade, Fisher Chemical) were used as stationary and mobile phases, respectively. THF flow rate was set at 1.0 ml/min. The autosampler, column and detector temperatures were set at 20, 25 and 25° C. for detection. The PGS control and PPGS samples were separately dissolved in THF with concentrations at 5.0 mg/ml per sample for analysis. A series of poly(styrene) standards with Mₚ ranged from 690 to 827000 Da were used to calibrate the molecular weights. All sample and standard solutions were filtered through 0.2 µm PTFE filter before testing. To each sample, the detection was replicated thrice.

SEM micrographics were obtained on Tescan-Mira3 SEM (TESCAN USA). The PPGS and PGS elastomers with thickness of approximately 1 mm were cut into strips. The freshly formed cross sections were coated with a thin layer of gold for SEM observation.

DSC analysis (Q1000 Modulated Differential Scanning Calorimeter, TA Instruments) was used to examine the thermal and crystalline properties. Samples with masses of 9.3 to 10.3 mg were cut from 1-mm thick PPGS and PGS sheets. Samples were respectively loaded in a standard Tzero pan with a lip. The test was performed with a heating rate at 10° C./min and the cooling rate at 5° C./min under nitrogen flow rate of 50 ml/min. The tests were recorded by first heating up from 25 to 200° C. and then cooling down to -70° C., followed by the second heating up to 200° C.

Surface hydrophobicity by water contact angle measurements. Water contact angle measurements were performed by the sessile drop method using a Rame-Hart 500 contact angle goniometer (Rame-Hart Inc., N.1) at room temperature. To each PPGS, two polymer films were made by casting the pre-polymer solution (0.5 ml, 30 % w/v in acetone) on two glass slides and air-dried for 24 h, followed by vacuum drying at 60° C. overnight. These films were then crosslinked at 150° C. for 24 h under reduced pressure and cooled down to room temperature before test. To each sample, 17 µl of water was dropped on the film surface and the contact angle was recorded at 50 second for comparison. The measurements were replicated five times on the two polymer films for each sample.

Preparation of PPGS and PGS elastomers. All PPGS and PGS control elastomers were made by crosslinking the pre-polymers at 150° C. for 24 h under reduced pressure (approximately 28 In. Hg vacuum) in a vacuum oven. The PPGS and PGS pre-polymers were separately dissolved in acetone to yield 30% w/v solutions for use. A rectangular silicone rubber mold (5 cm x 1.8 cm, 0.8 mm in thickness) was mounted on a glass slide for film preparation. To easily detach the crosslinked film from the mold, a thin layer of hyaluronic acid gel (1 wt.%) was applied to the glass surface and dried under reduced pressure overnight. 1 ml of each pre-polymer solution was transferred into the mold and air-dried for about 2 h each time until approximately 3.5 ml of each sample solution was subsequently transferred into the mold. The pre-polymer film was air-dried in a fume hood for approximately 24 h and further dried under reduced pressure at 60° C. overnight in the vacuum oven. The oven temperature was then increased to 150° C. for crosslinking to yield the PPGS and PGS elastomers. The crosslinked polymer films on the molds were then immersed into deionized water for 24 h and gently peeled off from the substrates. After airdrying, the transparent films with thickness of ca. 1 mm were obtained for mechanical and DSC tests.

Preparation of PGS and PPGS porous scaffolds. 30% w/v of PGS and PPGS pre-polymer in acetone solutions were prepared for use. NaCl salt was ground into particles with diameter between 75 and 150 µm. 4 g of the salt was evenly spread into a circular metal mold (D = 6 cm, thickness = 1 mm). The salt mold was fused for 1.5 h in a chamber with saturated water vapor at 37° C. The salt template was then dried at 80° C. overnight in a drying oven and cooled down to room temperature. Then 4.4 ml of each pre-polymer solution was slowly dropped onto the center of the salt template with pre-polymer to salt weight ratio of 1:3. In this way, each pre-polymer solution was evenly diffused throughout the salt template. After evaporation of solvent at room temperature overnight, these samples were further dried at 60° C. under reduced pressure for 3 h and then crosslinked at 150° C. for 24 h. After cooling down to room temperature, each sample was immersed in approximately 500 ml of deionized water for at least 48 h to wash away salt with replacement of deionized water every 24 h. The washed PGS and PPGS foams were freeze-dried for use.

Mechanical properties tests. Dog-bone samples were punched from crosslinked PGS and PPGS films using a dog-bone cutter (3.0 cm x 0.5 cm with a neck length of 10.5 mm and width of 1.5 mm). Uniaxial tensile and cyclic tensile tests were performed according to the standard method (ASTM D412) using Instron 5943 single column testing system equipped with 50N loading cell and Bluehill Universal software (INSTRON®, MA, USA). An elongation rate was set at 10 mm/min for the tensile tests and 30 mm/min for the cyclic tensile tests. The hysteresis tests were repeated thrice to obtain an average cyclic loading numbers for each sample. The uniaxial tensile tests for each sample were replicated five times to obtain mean values with standard deviations of the ultimate tensile strength (UTS), Young’s modulus (E) and strain at fracture (%).

In vitro degradation test. Disc samples were punched from the PPGS and PGS films and the porous scaffolds with a diameter of 6 mm and thickness of approximately 1 mm using a circular puncher. The initial mass of each sample was recorded as Mo. To each sample, 5 ml of 60 mM NaOH solution was added and the degradation test was set in an incubator at 37° C. for 6, 12, 18, 24, 48, 72 and 120 h. At the scheduled time points, the samples were collected, washed completely with deionized water and freeze-dried to record the residual mass as Mᵣ. To the porous scaffold samples, the degradation was similarly conducted at 37° C. in the 60 mM NaOH solution for 18 h and in PBS (pH 7.4) for one month. The degradation rate was calculated as degradation% = (M₀ - Mᵣ)x100/M₀.

In vitro cytocompatibility study. An in vitro cytotoxicity assay was performed on PGS and PPGS coating using HUVEC cells according to the standard protocol (ISO 10993). HLTVECs (passage 5) were cultured in endothelial cell growth medium containing 2% FBS and VEGF (EGM-2 BulletKit, CC-3156 & CC-4147, Lonza), 1% penicillin/streptomycin (Mediatech, Manassas, VA), and 1% L-glutamine at 37° C. with 5% CO₂ until sufficient quantities were obtained. The cells were diluted in cell media to 1x10⁴ cell ml⁻¹ for use.

0.25 % w/v of PGS or PPGS pre-polymer in acetone solutions were prepared and 20 µl of each solution was evenly spread on a 12 mm diameter circular cover glass. A pre-polymer coating with a thickness of approximately 200 nm was formed on the cover glass. The slide was air-dried in a fume hood overnight and then crosslinked in a vacuum oven at 150° C. for 24 h. The coated slides were placed into a 24-well culture plate with the coating layer orientated upward. The coating was sterilized by UV exposure for 1 h and subsequently washed with DPBS 1x (pH 7.4) and cell media (20 min (min = minute(s)) per wash). Then 1x10⁴ cells were seeded on the coating with 1 ml of cell media per well. The plate was incubated at 37° C. with 5% CO₂. After incubation for 24 and 48 h, cell metabolism (MTT assay, n=4) was determined by a CellTiter-Blue Cell Viability Assay Kit (Promega, Madison, WI). Live/dead assay (n=4) was performed using LIVE/DEAD Viability/Cytotoxicity Kit (Invitrogen, Carlsbad, CA). The absorbance and fluorescence were recorded using the SpectraMax M3 microplate reader (Molecular Devices, LLC. USA). The fluorescent microscopic images of the live/dead assay were recorded using Nikon ECLIPSE Ti2 microscope (Nikon Instruments Inc., NY, USA). The cells cultured in a 24-well tissue culture polystyrene plate (TCPS) were used as a control.

Statistical analysis. Statistical analysis was performed using one-way ANOVA with post test of Bonferroni’s multiple comparison test. Ap value < 0.05 is considered significantly different. Data represent the mean value ± standard deviation (SD).

### Example 2

This example provides a description of a PGS polymer of the present disclosure, making the PGS polymer, and characterization of the PGS polymer.

Synthesis of benzoate-functionalized poly(glycerol sebacate) (BPGS). All chemicals were used as received. When synthesizing benzoate-functionalized poly(glycerol sebacate), the protocol is slightly modified because of likely more reactive nature of benzoic anhydride. For example, poly(glycerol sebacate) (Regenerez®, Secant Group LLC, PA) was reacted with theoretical 20 mol.% of benzoic anhydride (TCI, >97.0%) to make the benzoate-functionalized poly(glycerol sebacate) (BPGS). Specifically, 25 mmol of PGS (6.46 g) (based on the repeat units) and 5 mmol of benzoic anhydride (1.13 g) were mixed in 50 ml of 1,4-dioxane anhydrous (Spectrum, 99.8%) in a round bottom flask. Then 10 mmol of trimethylamine (1.4 ml) was added to the mixture. The mixture was dissolved at 50° C. for approximately 15 min (min = minute(s)). The reaction was then continued at 90° C. for 11 h (h = hour(s)) and at room temperature for approximately 9 h with magnetically stirring under nitrogen atmosphere. After reaction, the viscous product solution was precipitated in 900 ml of hexanes (Pharmco, ACS grade) with gentle magnetic stirring for 3 h. The supernatant was decanted and the viscous residue was dissolved in 80 ml of acetone (Pharmco, ACS grade), followed by adding approximately 900 ml of hexanes again for precipitation with magnetic stirring overnight. The viscous sediment was washed with approximately 300 ml of hexanes again. The viscous product was collected and dried under reduced pressure.

Compared to palmitate-functionalized PGS at the same theoretic content (20 mol.%), benzoate-functionalized PGS is more adhesive and difficult to dissolve in acetone and dioxane.

Although the present disclosure has been described with respect to one or more particular examples, it will be understood that other examples of the present disclosure may be made without departing from the scope of the present disclosure.

1. A poly(glycerol sebacate) (PGS) polymer comprising pendant aliphatic carboxylate groups and/or pendant aryl carboxylate groups, wherein each pendant aliphatic carboxylate group and/or pendant aryl carboxylate group is covalently bound to the glycerol group of the glycerol sebacate backbone of the polymer.

2. The poly(glycerol sebacate) polymer of claim 1, wherein in the poly(glycerol sebacate) polymer comprises the following structure: 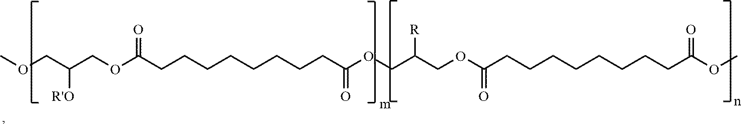
wherein R is a pendant group independently chosen from aliphatic carboxylate groups and aryl carboxylate groups,
R′ is independently chosen from hydrogen group, sebacate group, oligo(glycerol sebacate) groups, and poly(glycerol sebacate) groups,
m is 0 to 99, and
n is 1 to 100, wherein m an n are molar % values.

3. The poly(glycerol sebacate) polymer of claim 1, wherein 1 to 100% of the glycerol groups of the poly(glycerol sebacate) polymer have a pendant aliphatic carboxylate group and/or pendant aryl carboxylate group.

4. The poly(glycerol sebacate) polymer of claim 1, wherein the pendant aliphatic carboxylate groups are independently chosen from C₃ to C₄₀ aliphatic carboxylate groups.

5. The poly(glycerol sebacate) polymer of claim 1, wherein the pendant aryl carboxylate groups are independently chosen from C₆ to C₁₂ aryl carboxylate groups.

6. The poly(glycerol sebacate) polymer of claim 1, wherein the poly(glycerol sebacate) polymer is at least partially crosslinked.

7. A polymeric material comprising a plurality of glycerol sebacate groups, wherein at least a portion of the individual glycerol sebacate groups have a pendant aliphatic carboxylate group and/or pendant aryl carboxylate group covalently bound to the glycerol group of the glycerol sebacate group.

8. The polymeric material of claim 7, wherein in the polymeric material comprises the following structure: 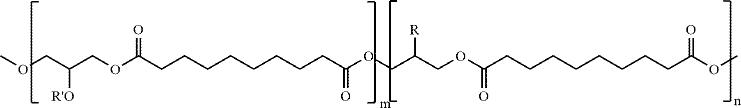
,
wherein R is a pendant group independently chosen from aliphatic carboxylate groups and aryl carboxylate groups,
R′ is independently chosen from hydrogen group, sebacate group, oligo(glycerol sebacate) group, and poly(glycerol sebacate) group, with the proviso that not each R′ is a hydrogen group,
m is 0 to 99, and
n is 1 to 100, wherein m an n are molar % values.

9. The polymeric material of claim 7, wherein the polymeric material is copolymer.

10. The polymeric material of claim 9, wherein the copolymer is a block copolymer comprising one or more poly(glycerol sebacate) block(s) formed from a plurality of the glycerol sebacate groups.

11. The polymeric material of claim 9, wherein the copolymer is a block copolymer comprising one or more additional block(s) chosen from hydrophilic blocks, hydrophobic blocks, and combinations thereof.

12. The polymeric material of claim 7, wherein the polymeric material is at least partially crosslinked.

13. A fiber comprising one or more poly(glycerol sebacate)(s), each poly(glycerol sebacate), comprising pendant aliphatic carboxylate groups and/or pendant aryl carboxylate groups, and each pendant aliphatic carboxylate group and/or pendant aryl carboxylate group is covalently bound to the glycerol group of the glycerol sebacate backbone of the polymer, and/or
one or more polymeric material(s), each polymeric material comprising a plurality of glycerol sebacate groups, wherein at least a portion of the individual glycerol sebacate groups have a pendant aliphatic carboxylate group and/or pendant aryl carboxylate group covalently bound to the glycerol group of the glycerol sebacate group.

14. The fiber of claim 13, wherein the fiber comprises one or more other polymer(s) and/or one or more other polymeric material(s) chosen from polylactic acids (PLAs), polyglycolic acids (PGAs), PLGAs, poly(caprolactone)s (PCLs), polyethylene glycols (PEGs), polyethylene terephthalates (PETs), polypropylenes, polyethylenes, nylons, polystyrenes, and combinations thereof.

15. The fiber of claim 13, polymeric material of claim 7, wherein the one or more poly(glycerol sebacate)(s) and/or one or more polymer materials is/are at least partially crosslinked.

16. A material comprising a plurality of one or more fiber(s) of claim 13.

17. The material of claim 13, wherein the material is a fabric.

18. The material of claim 14, wherein the material comprises one or more other fiber(s) chosen from polylactic acid (PLA) fibers, polyethylene glycol (PEG) fibers, PLGA fibers, poly(lactide-co-caprolactone) (PLCL) fibers, polyglycolic acid (PGA) fibers, PLGA fibers, poly(caprolactone) (PCL) fibers, polyethylene terephthalate (PET) fibers, polypropylene fibers, polyethylene fibers, nylon fibers, polystyrene fibers, and combinations thereof.

19. A tissue graft comprising one or more material(s) of claim 16.

20. The tissue graft of claim 19, wherein the tissue graft is a vascular graft.

21. The tissue graft of claim 20, wherein the vascular graft is an arterial graft.

22. The tissue graft of claim 21, wherein the arterial graft is a small artery graft.

23. The tissue graft of claim 22, wherein the small artery graft has a lumen diameter of 6 mm or less.
